(19) United States
(12) Patent Application Publication
(10) Pub. No.: US 2007/0225240 A1
(43) Pub. Date: Sep. 27, 2007

(54) Screening Assay and Treatment

(76) Inventors: Xin Lu, London (GB); Nadia Godin-Heymann, Cambridge, MA (US)

Correspondence Address: KLARQUIST SPARKMAN, LLP, 121 SW SALMON STREET, SUITE 1600, PORTLAND, OR, 97204, US

(21) Appl. No.: 10/581,569

(22) PCT Filed: Sep. 13, 2004
(86) PCT No.: PCT/GB04/03899

§ 371 Date: Jan. 16, 2007

Related U.S. Application Data
(60) Provisional application No. 60/554,852, filed on Mar. 19, 2004.

(30) Foreign Application Priority Data
Dec. 4, 2003 (GB) 0328106.0
Feb. 6, 2004 (GB) 0404242.0

(51) Int. Cl. A61K 31/7088; A01K 67/027; C07K 16/32; C12N 15/63; C12N 5/16; G01N 33/574

(52) U.S. Cl. 514/44; 435/320.1; 435/325; 435/7.2; 435/7.23; 530/387.9; 530/388.1; 800/13; 800/14; 800/17; 800/18

(57) ABSTRACT

The invention relates to screening assays for the identification of agents which modulate the activity of polypeptides which affect the apoptotic activity of the tumour suppressor protein p53 and including gene therapy vectors comprising p53 and antibodies that bind phosphorylated epitopes.

The invention relates to an screening assay for the identification of agents which modulate the activity of polypeptides which affect the apoptotic activity of the tumour suppressor protein p53 and including gene therapy vectors comprising p53.

Tumour suppressor genes encode proteins which function to inhibit cell growth or division and are therefore important with respect to maintaining proliferation, growth and differentiation of normal cells. Mutations in tumour suppressor genes result in abnormal cell-cycle progression whereby the normal cell-cycle check points which arrest the cell-cycle, when, for example, DNA is damaged, are ignored and damaged cells divide uncontrollably. The products of tumour suppressor genes function in all parts of the cell (e.g. cell surface, cytoplasm, nucleus) to prevent the passage of damaged cells through the cell-cycle (i.e. G1, S, G2, M and cytokinesis). Arguably the tumour suppressor gene which has been the subject of the most intense research is p53. p53 encodes a protein which functions as a transcription factor and is a key regulator of the cell division cycle. It was discovered as a protein shown to bind with affinity to the SV40 large T antigen. The p53 gene encodes a 393 amino acid polypeptide with a molecular weight of 53 kDa.

We have described a family of proteins in WO02/12325 which function to enhance the apoptotic activity of p53. ASPP1 and ASPP2 selectively interact with p53 to enhance the apoptotic function of p53 at p53 responsive promoters to promote apoptosis in vivo. We herein describe the interaction of ASPP family members with the oncogene Ras. ASPP 1 and 2 are also phophoproteins.

Ras oncogenes are frequently activated by mutation or over expression in many human tumours. For example, approximately 95% of pancreatic tumours contain so called K-Ras mutations. Ras oncogenes are believed to exert their effect by over-riding the normal cell-cycle control mechanisms by activating protein kinases (e.g. Raf, Mek, Erk kinase pathways) which regulate the function of cell-cycle cyclins which promote the proliferation of eukaryotic cells.

In its inactive state Ras is bound to GDP. The activation of Ras by growth factors results in exchange of GDP for GTP and a consequent change in the conformation of Ras to an activated form. In vitro, Ras has an intrinsic GTPase activity which becomes active when growth factor stimulation is removed and returns Ras to its GDP bound state. Ras is also a post-translationally modified protein and it is this modification which facilitates the localisation of Ras to the cell membrane and allows Ras to receive growth factor signals. The post-translational modification is farnesylation which results in the alkylation of cysteine residues in a conserved motif “CAAX”. Ras has three CAAX motifs located in the C-terminus of the protein and it has been shown that inhibition of the farnesylation reaction of Ras blocks its processing and thereby inactivates the protein. The farnesylation reaction has been a target for the rational design of agents which inhibit the reaction thereby preventing the localisation of Ras at its site of action, the inner cell membrane. However, the farnesylation reaction is more complicated that was first seemed. For example, H Ras is exclusively modified by farnsyltransferase whereas K-Ras and N-Ras can also be modified by gerangylgerangyltransferase. This has meant that there is a continuing need to identify new targets which can modulate Ras activity or oncogenic Ras activity, either directly or indirectly,

We have identified ASPP 1/2 as Ras binding targets. The binding domain in ASPP1/2 is the amino terminus of the protein. We also show that Ras activates ASPP through the MAPK and Raf CX pathway and that dephosphorylation by phosphatase action is an important factor regulating ASPP action. The interaction of these protein factors with ASPP provides an opportunity to identify agents which enhance or inhibit the action of ASPP on p53 mediated apoptosis.

According to an aspect of the invention there is provided method for the identification of agents which modulate the interaction of the proto-oncogene/oncogene Ras with the p53 binding protein family ASPP, either directly or indirectly.

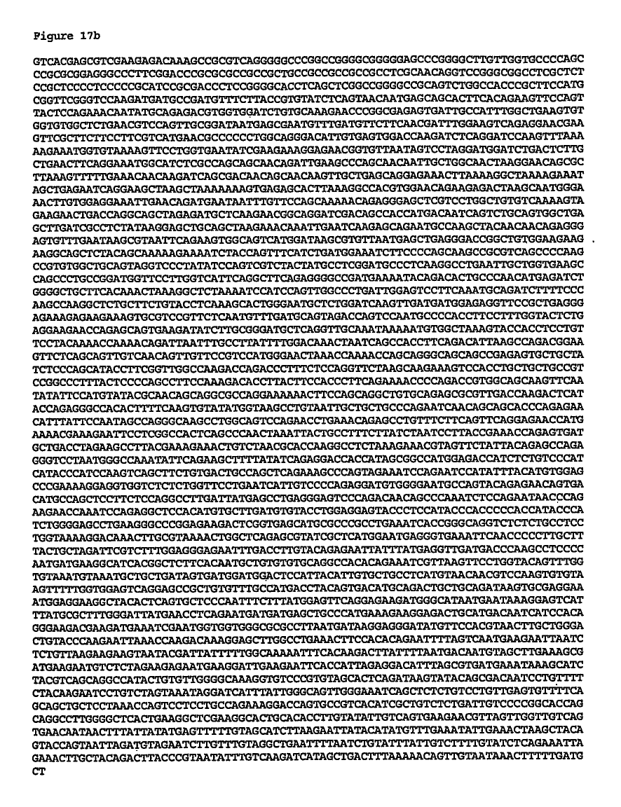

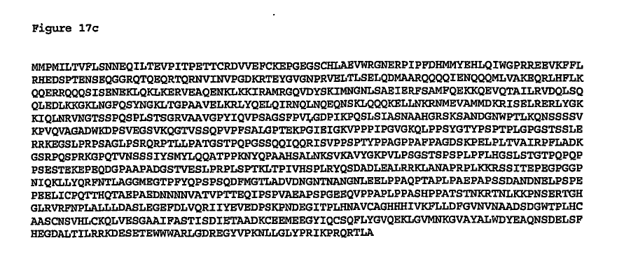

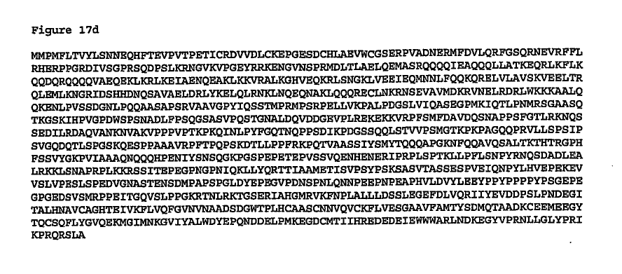

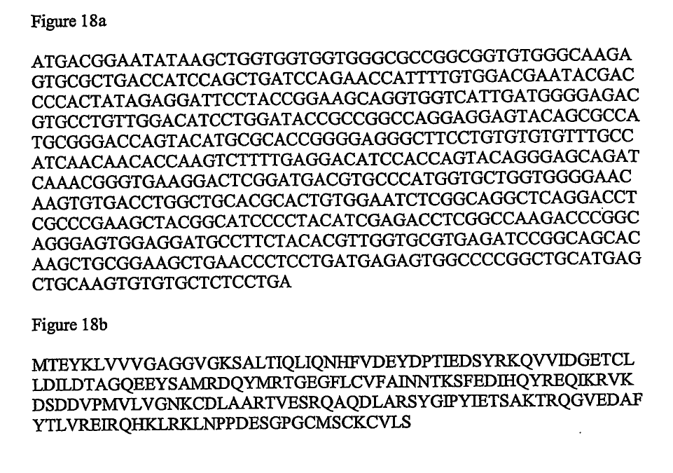

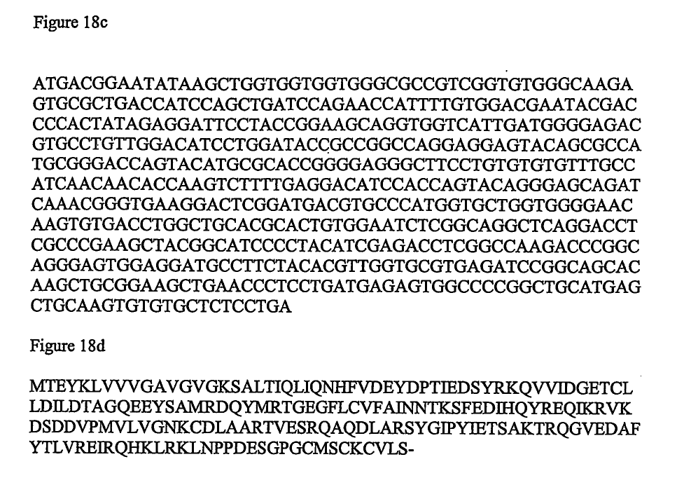

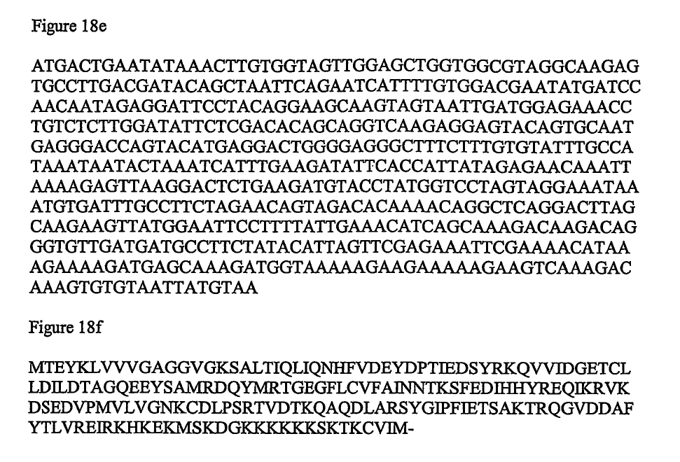

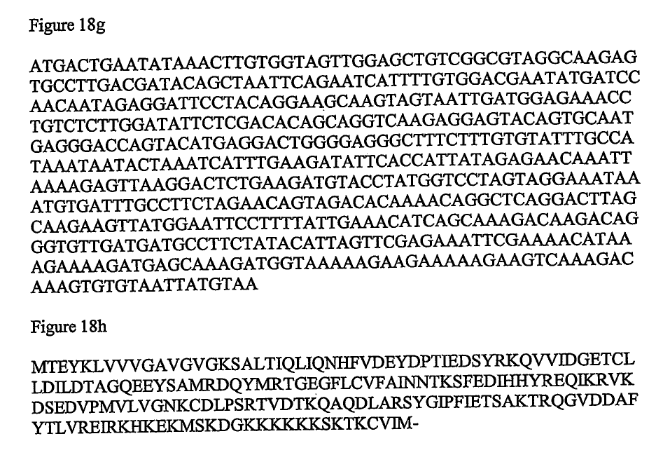

According to an aspect of the invention there is provided a screening method for the identification of agents which modulate the interaction of a first polypeptide encoded by a nucleic acid molecule selected from the group consisting of:
a) a polypeptide encoded by a nucleic acid molecule comprising a nucleic acid sequence as represented in FIG. 17a or 17b;
b) a polypeptide encoded by a nucleic acid molecule which hybridises to the nucleic acid molecule in (a) and which enhances the pro-apoptotic activity of p53;
c) a polypeptide encoded by a nucleic acid molecule consisting of a nucleic acid sequence that is degenerate as a result of the genetic code to a nucleic acid molecule as defined in (a) and (b); with a second polypeptide selected from the group consisting of:
d) a polypeptide encoded by a nucleic acid molecule comprising a nucleic acid sequence as represented in FIG. 18a, 18c, 18e or 18g;
e) a polypeptide encoded by a nucleic acid molecule which hybridises to the nucleic acid molecule in (d) above and which has the activity associated with Ras, or a variant Ras polypeptide;
f) a polypeptide encoded by a nucleic acid molecule consisting of a nucleic acid sequence that is degenerate as a result of the genetic code to a nucleic acid molecule as defined in (d) and (e); comprising,
i) forming a preparation comprising said first and second polypeptide;
ii) adding at least one candidate agent to be tested; and
iii) determining the effect, or not, of said agent on the interaction of said first polypeptide with said second polypeptide.

In a preferred method of the invention said first polypeptide is represented by the amino acid sequence as shown in FIG. 17c or 17d, or a variant polypeptide wherein said variant polypeptide sequence has been altered by addition, substitution or deletion of at least one amino acid residue.

A variant polypeptide may differ in amino acid sequence by one or more substitutions, additions, deletions, truncations which may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and asparatic acid; c) asparagine and glutamine d) arginine and lysine; e) isoleucine, leucine, methionine and valine and f) phenylalanine, tyrosine and tryptophan.

In addition, the invention features polypeptide sequences having at least 75% identity with the polypeptide sequence as hereindisclosed, or fragments and functionally equivalent polypeptides thereof. In one embodiment, the polypeptides have at least 85% identity, more preferably at least 90% identity, even more preferably at least 95% identity, still more preferably at least 97% identity, and most preferably at least 99% identity with the amino acid sequence illustrated herein.

In a further preferred method of the invention said first polypeptide comprises the amino acid sequence +1 to +120 of the sequence shown in FIG. 17c and 17d Preferably said polypeptide consists of the amino acid sequence +1 to +120 of the sequence shown in FIG. 17c or 17d.

In a further preferred method of the invention said second polypeptide is represented by the amino acid sequence shown in FIG. 18b, 18d, 18f or 18h, or a variant polypeptide wherein said variant polypeptide sequence has been altered by addition, substitution or deletion of at least one amino acid residue.

In a preferred method of the invention said second polypeptide is modified at amino acid residue 12. Preferably said modification is the substitution of amino acid 12 for amino acid valine. Preferably said second polypeptide is K-RasV12. Alternatively said polypeptide is H-RasV12.

In a yet further preferred method of the invention said second polypeptide is modified at amino acid residue 17. Preferably said modification is substitution of serine for asparagine at amino acid residue 17.

In a further preferred method of the invention said first and second polypeptides are expressed by a cell.

In a preferred method of the invention said cell is a cell transfected with at least one nucleic acid molecule(s) which encodes said first and/or second polypeptides.

Preferably the expression of said nucleic acid molecule(s) is regulatable.

In a preferred method of the invention said cell is a cancer cell.

In a yet further preferred method of the invention said cell is part of a transgenic animal wherein the genome of said animal has been modified to include nucleic acid molecules which encode first and second polypeptides. Preferably said nucleic acid molecules are expressed in a specific cell/tissue.

In a yet still further preferred method of the invention said preparation includes at least one chemotherapeutic agent.

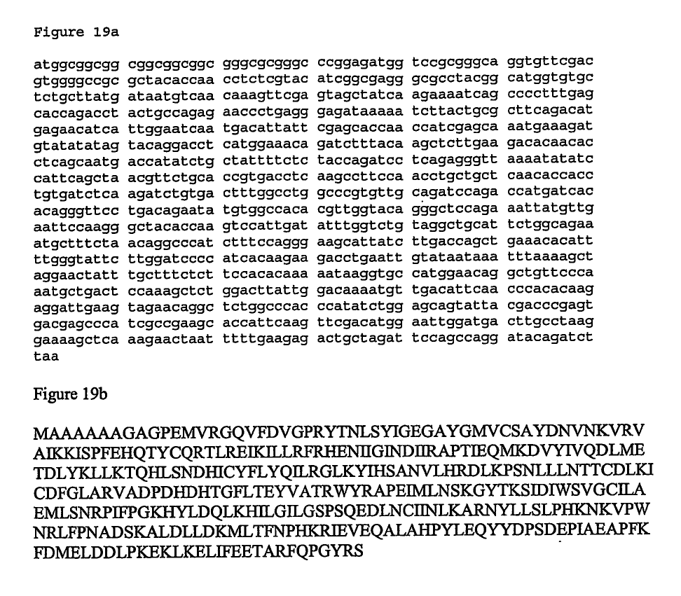

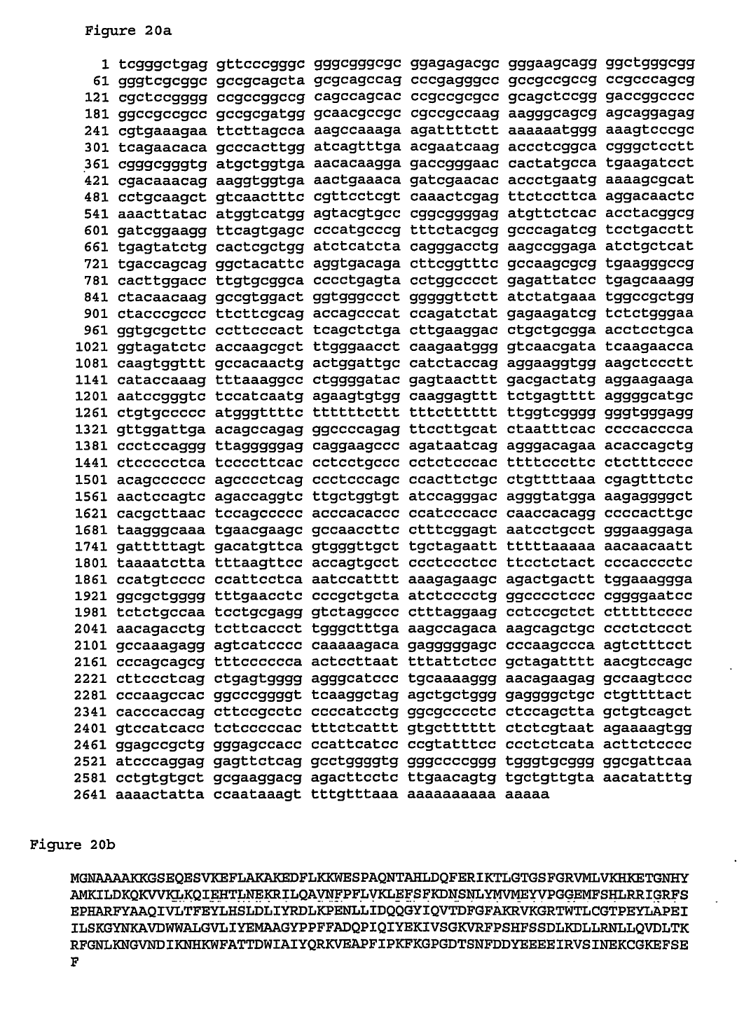

According to an aspect of the invention there is provided a screening method for the identification of agents which modulate the phosphorylation of a first polypeptide encoded by a nucleic acid molecule selected from the group consisting of:
a) a polypeptide encoded by a nucleic acid molecule comprising a nucleic acid sequence as represented in FIG. 17a or 17b;
b) a polypeptide encoded by a nucleic acid molecule which hybridises to the nucleic acid molecule in (a) and which enhances the pro-apoptotic activity of p53;
c) a polypeptide encoded by a nucleic acid molecule consisting of a nucleic acid sequence that is degenerate as a result of the genetic code to a nucleic acid molecule as defined in (a) and (b); with a second polypeptide selected from the group consisting of:
d) a polypeptide encoded by a nucleic acid molecule comprising a nucleic acid sequence as represented in FIG. 19 or 20;
e) a polypeptide encoded by a nucleic acid molecule which hybridises to the nucleic acid molecule in (d) above and which has protein kinase activity;
f) a polypeptide encoded by a nucleic acid molecule consisting of a nucleic acid sequence that is degenerate as a result of the genetic code to a nucleic acid molecule as defined in (d) and (e); comprising,
i) forming a preparation comprising said first and second polypeptide;
ii) adding at least one candidate agent to be tested; and
iii) determining the effect, or not, of said agent on the phosphorylation state of said first polypeptide.

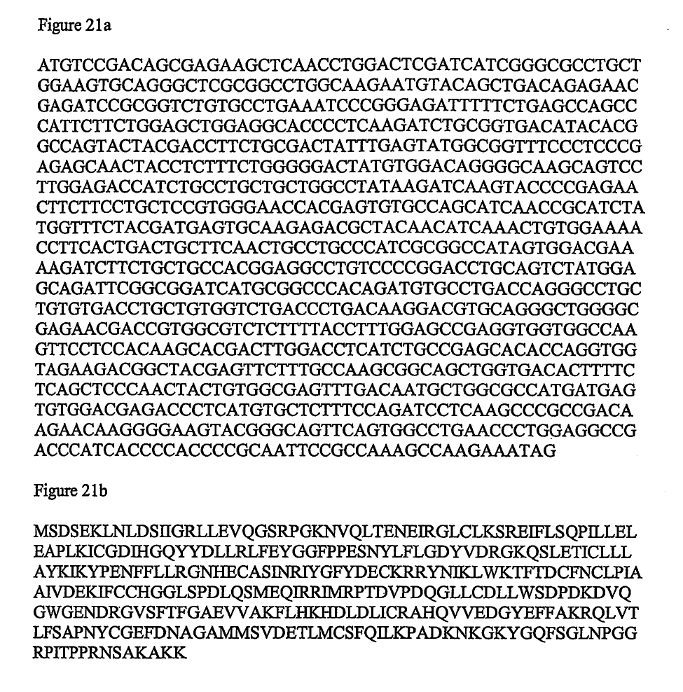

According to a further aspect of the invention there is provided a screening method for the identification of agents which modulate the phosphorylation state of a first polypeptide encoded by a nucleic acid molecule selected from the group consisting of:
a) a polypeptide encoded by a nucleic acid molecule comprising a nucleic acid sequence as represented in FIG. 17a or 17b;
b) a polypeptide encoded by a nucleic acid molecule which hybridises to the nucleic acid molecule in (a) and which enhances the pro-apoptotic activity of p53;
c) a polypeptide encoded by a nucleic acid molecule consisting of a nucleic acid sequence that is degenerate as a result of the genetic code to a nucleic acid molecule as defined in (a) and (b); with a second polypeptide selected from the group consisting of:
d) a polypeptide encoded by a nucleic acid molecule comprising a nucleic acid sequence as represented in FIG. 21;
e) a polypeptide encoded by a nucleic acid molecule which hybridises to the nucleic acid molecule in (d) above and which has protein phosphatase activity;
f) a polypeptide encoded by a nucleic acid molecule consisting of a nucleic acid sequence that is degenerate as a result of the genetic code to a nucleic acid molecule as defined in (d) and (e); comprising,
i) forming a preparation comprising said first and second polypeptide;
ii) adding at least one candidate agent to be tested; and
iii) determining the effect, or not, of said agent on the phosphorylation state of said first polypeptide.

In a preferred method of the invention said agent is a polypeptide.

In a preferred method of the invention said polypeptide is an antibody, or active binding fragment thereof.

Preferably said antibody or binding fragment is a monoclonal antibody.

Antibodies or immunoglobulins (Ig) are a class of structurally related proteins consisting of two pairs of polypeptide chains, one pair of light (L) (low molecular weight) chain (κ or λ), and one pair of heavy (H) chains (γ, α, μ, δ and ε), all four linked together by disulphide bonds. Both H and L chains have regions that contribute to the binding of antigen and that are highly variable from one Ig molecule to another. In addition, H and L chains contain regions that are non-variable or constant. The L chains consist of two domains. The carboxy-terminal domain is essentially identical among L chains of a given type and is referred to as the “constant” (C) region. The amino terminal domain varies from L chain to L chain and contributes to the binding site of the antibody. Because of its variability, it is referred to as the “variable” (V) region. The variable region contains complementarity determining regions or CDR's which form an antigen binding pocket. The binding pockets comprise H and L variable regions which contribute to antigen recognition. It is possible to create single variable regions, so called single chain antibody variable region fragments (scFv's). If a hybridoma exists for a specific monoclonal antibody it is well within the knowledge of the skilled person to isolate scFv's from mRNA extracted from said hybridoma via RT PCR. Alternatively, phage display screening can be undertaken to identify clones expressing scFv's.

Alternatively said fragments are “domain antibody fragments”. Domain antibodies are the smallest binding part of an antibody (approximately 13 kDa). Examples of this technology is disclosed in U.S. Pat. No. 6,248,516, U.S. Pat. No. 6,291,158, U.S. Pat. No. 6,127, 197 and EP0368684 which are all incorporated by reference in their entirety.

In a preferred method of the invention said antibody fragment is a single chain antibody variable region fragment.

In a further preferred embodiment of the invention said antibody is a humanised or chimeric antibody.

A chimeric antibody is produced by recombinant methods to contain the variable region of an antibody with an invariant or constant region of a human antibody. A humanised antibody is produced by recombinant methods to combine the complementarity determining regions (CDRs) of an antibody with both the constant (C) regions and the framework regions from the variable (V) regions of a human antibody.

Chimeric antibodies are recombinant antibodies in which all of the V-regions of a mouse or rat antibody are combined with human antibody C-regions. Humanised antibodies are recombinant hybrid antibodies which fuse the complimentarity determining regions from a rodent antibody V-region with the framework regions from the human antibody V-regions. The C-regions from the human antibody are also used. The complimentarily determining regions (CDRs) are the regions within the N-terminal domain of both the heavy and light chain of the antibody to where the majority of the variation of the V-region is restricted. These regions form loops at the surface of the antibody molecule. These loops provide the binding surface between the antibody and antigen.

Antibodies from non-human animals provoke an immune response to the foreign antibody and its removal from the circulation. Both chimeric and humanised antibodies have reduced antigenicity when injected to a human subject because there is a reduced amount of rodent (i.e. foreign) antibody within the recombinant hybrid antibody, while the human antibody regions do not elicit an immune response. This results in a weaker immune response and a decrease in the clearance of the antibody. This is clearly desirable when using therapeutic antibodies in the treatment of human diseases. Humanised antibodies are designed to have less “foreign” antibody regions and are therefore thought to be less immunogenic than chimeric antibodies.

In a further preferred method of the invention said agent is a peptide, preferably a modified peptide.

It will be apparent to one skilled in the art that modification to the amino acid sequence of peptides which modulate the interaction of ASPP family members with polypeptides involved in regulating ASPP activity could enhance the binding and/or stability of the peptide with respect to its target sequence. In addition, modification of the peptide may also increase the in vivo stability of the peptide thereby reducing the effective amount of peptide necessary to inhibit an interaction. This would advantageously reduce undesirable side effects which may result in vivo. Modifications include, by example and not by way of limitation, acetylation and amidation.

In a preferred method of the invention said peptide is acetylated. Preferably said acetylation is to the amino terminus of said peptide.

In a further preferred method of the invention said peptide is amidated. Preferably said amidation is to the carboxyl-terminus of said peptide.

In a further preferred method of the invention said peptide is modified by both acetylation and amidation.

Alternatively, or preferably, said modification includes the use of modified amino acids in the production of recombinant or synthetic forms of peptides. It will be apparent to one skilled in the art that modified amino acids include, by way of example and not by way of limitation, 4-hydroxyproline, 5-hydroxylysine, N⁶-acetyllysine, N⁶-methyllysine, N⁶,N⁶-dimethyllysine, N⁶,N⁶,N⁶-trimethyllysine, cyclohexyalanine, D-amino acids, ornithine. Other modifications include amino acids with a C₂, C₃ or C₄ alkyl R group optionally substituted by 1, 2 or 3 substituents selected from halo (e.g. F, Br, I), hydroxy or C₁-C₄ alkoxy.

Altematively, peptides could be modified by, for example, cyclisation. Cyclisation is known in the art, (see Scott et al Chem Biol (2001), 8:801-815; Gellerman et al J. Peptide Res (2001), 57: 277-291; Dutta et al J. Peptide Res (2000), 8: 398412; Ngoka and Gross J Amer Soc Mass Spec (1999), 10:360-363.

In a preferred method of the invention peptides according to the invention are modified by cyclisation.

In a further preferred method of the invention said agent is an aptamer.

Nucleic acids have both linear sequence structure and a three dimensional structure which in part is determined by the linear sequence and also the environment in which these molecules are located. Conventional therapeutic molecules are small molecules, for example, peptides, polypeptides, or antibodies, which bind target molecules to produce an agonistic or antagonistic effects. It has become apparent that nucleic acid molecules also have potential with respect to providing agents with the requisite binding properties which may have therapeutic utility. These nucleic acid molecules are typically referred to as aptamers. Aptamers are small, usually stabilised, nucleic acid molecules, which comprise a binding domain for a target molecule. A screening method to identify aptamers is described in U.S. Pat. No. 5,270,163 which is incorporated by reference. Aptamers are typically oligonucleotides which may be single stranded oligodeoxynucleotides, oligoribonucleotides, or modified oligodeoxynucleotide or oligoribonucleotides.

The term “modified” encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2-O-alkyl; 2-O-allyl; 2′-S-alkyl; 2′-S-allyl; 2′-fluoro-; 2′-halo or 2; azido-ribose, carbocyclic sugar analogues a-anomeric sugars; epimeric sugars such as arabinose, xyloses or lyxoses, pyranose sugars, furanose sugars, and sedoheptulose.

Modified nucleotides are known in the art and include, by example and not by way of limitation, alkylated purines and/or pyrimidines; acylated purines and/or pyrimidines; or other heterocycles. These classes of pyrimidines and purines are known in the art and include, pseudoisocytosine; N4, N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2-methylthio-N6-isopentenyladenine; uracil-5-oxyacetic acid methyl ester; psueouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6,-diaminopurine; methylpsuedouracil; 1-methylguanine; 1-methylcytosine.

The aptamers of the invention are synthesised using conventional phosphodiester linked nucleotides and synthesised using standard solid or solution phase synthesis techniques which are known in the art. Linkages between nucleotides may use alternative linking molecules. For example, linking groups of the formula P(O)S, (thioate); P(S)S, (dithioate); P(O)NR′2; P(O)R′; P(O)OR6; CO; or CONR′2 wherein R is H (or a salt) or alkyl (1-12C) and R6 is alkyl (1-9C) is joined to adjacent nucleotides through —O— or —S—. The binding of aptamers to a target polypeptide is readily tested by assays hereindisclosed.

According to a further aspect of the invention there is provided an antibody, or active binding fragment thereof, wherein said antibody or fragment specifically binds a polypeptide encoded by a nucleic acid molecule selected from the group consisting of:
a) a polypeptide encoded by a nucleic acid molecule comprising a nucleic acid sequence as represented in FIG. 17a or 17b;
b) a polypeptide encoded by a nucleic acid molecule which hybridises to the nucleic acid molecule in (a) and which enhances the pro-apoptotic activity of p53;
c) a polypeptide encoded by a nucleic acid molecule consisting of a nucleic acid sequence that is degenerate as a result of the genetic code to a nucleic acid molecule as defined in (a) and (b), wherein said antibody binds a phosphorylated epitope.

In a preferred embodiment of the invention said antibody is a monoclonal antibody.

In a preferred embodiment of the invention said antibody fragment is a single chain antibody fragment or a domain antibody.

In a preferred embodiment of the invention said phosphorylated epitope comprises amino acid residue 671 of the amino acid sequence as shown in FIG. 17c.

In an alternative preferred embodiment of the invention said phosphorylated epitope comprises amino acid residue 698 of the amino acid sequence shown in FIG. 17d.

In a further preferred embodiment of the invention said phosphorylated epitope comprises amino acid residue 746 of the amino acid sequence as shown in FIG. 17c.

In a yet preferred embodiment of the invention said phosphorylated epitope comprises amino acid residue 827 of the amino acid sequence shown in FIG. 17d.

According to a further aspect of the invention there is provided a cell transfected with at least one nucleic acid molecule wherein the genome of said cell is modified to include at least one copy of a nucleic acid molecule encoding a polypeptide selected from the group consisting of:
a) a polypeptide encoded by a nucleic acid molecule comprising a nucleic acid sequence as represented in FIG. 17a or 17b;
b) a polypeptide encoded by a nucleic acid molecule which hybridises to the nucleic acid molecule in (a) and which enhances the pro-apoptotic activity of p53;
c) a polypeptide encoded by a nucleic acid molecule consisting of a nucleic acid sequence that is degenerate as a result of the genetic code to a nucleic acid molecule as defined in (a) and (b); and at least one copy of a nucleic acid molecule encoding a polypeptide selected from the group consisting of:
d) a polypeptide encoded by a nucleic acid molecule comprising a nucleic acid sequence as represented in FIG. 18a, 18c, 18e, or 18g;
e) a polypeptide encoded by a nucleic acid molecule which hybridises to the nucleic acid molecule in (d) above and which has the activity associated with Ras or a variant Ras polypeptide;
f) a polypeptide encoded by a nucleic acid molecule consisting of a nucleic acid sequence that is degenerate as a result of the genetic code to a nucleic acid molecule as defined in (d) and (e) wherein said cell is adapted for the regulated expression of said nucleic acid molecules.

According to a further aspect of the invention there is provided a cell transfected with at least one nucleic acid molecule wherein the genome of said cell is modified to include at least one copy of a nucleic acid molecule encoding a polypeptide selected from the group consisting of:
a) a polypeptide encoded by a nucleic acid molecule comprising a nucleic acid sequence as represented in FIG. 17a or 17b;
b) a polypeptide encoded by a nucleic acid molecule which hybridises to the nucleic acid molecule in (a) and which enhances the pro-apoptotic activity of p53;
c) a polypeptide encoded by a nucleic acid molecule consisting of a nucleic acid sequence that is degenerate as a result of the genetic code to a nucleic acid molecule as defined in (a) and (b) and at least one copy of a nucleic acid molecule encoding a polypeptide selected from the group consisting of;
d) a polypeptide encoded by a nucleic acid molecule comprising a nucleic acid sequence as represented in FIG. 19a or 20a;
e) a polypeptide encoded by a nucleic acid molecule which hybridises to the nucleic acid molecule in (d) above and which has protein kinase activity;
f) a polypeptide encoded by a nucleic acid molecule consisting of a nucleic acid sequence that is degenerate as a result of the genetic code to a nucleic acid molecule as defined in (d) and (e) wherein said cell is adapted for the regulated expression of said nucleic acid molecules.

According to a yet further aspect of the invention there is provided a cell transfected with at least one nucleic acid molecule wherein the genome of said cell is modified to include at least one copy of a nucleic acid molecule encoding a polypeptide selected from the group consisting of:
a) a polypeptide encoded by a nucleic acid molecule comprising a nucleic acid sequence as represented in FIG. 17a or 17b;
b) a polypeptide encoded by a nucleic acid molecule which hybridises to the nucleic acid molecule in (a) and which enhances the pro-apoptotic activity of p53;
c) a polypeptide encoded by a nucleic acid molecule consisting of a nucleic acid sequence that is degenerate as a result of the genetic code to a nucleic acid molecule as defined in (a) and (b) and at least one copy of a nucleic acid molecule encoding a polypeptide selected from the group consisting of;
d) a polypeptide encoded by a nucleic acid molecule comprising a nucleic acid sequence as represented in FIG. 21a;
e) a polypeptide encoded by a nucleic acid molecule which hybridises to the nucleic acid molecule in (d) above and which has protein kinase activity;
f) a polypeptide encoded by a nucleic acid molecule consisting of a nucleic acid sequence that is degenerate as a result of the genetic code to a nucleic acid molecule as defined in (d) and (e) wherein said cell is adapted for the regulated expression of said nucleic acid molecules.

In a preferred embodiment of the invention said cell further comprises a nucleic acid molecule which includes a reporter gene to monitor the activity of said pro-apoptotic polypeptide(s).

In a preferred embodiment of the invention said cell is a cancer cell.

According to a yet further aspect of the invention there is provided a non-human transgenic animal comprising at least one cell according to the invention.

In a preferred embodiment of the invention said non-human animal is a non-human primate.

In a further preferred embodiment of the invention said transgenic animal is a rodent, preferably a mouse, rat or hamster.

In an alternative preferred embodiment of the invention said transgenic animal is a pig.

It is well within the knowledge of the skilled person to successfully generate both a heterozygous and homozygous transgenic animals with intergation of a desired nucleic acid encoding a particular gene or combination of genes using modified embryonic stem cells. These same steps can be easily applicable to species other than for example, mice. For example, Ware et al teaches an embryonic stem cell culture condition amenable for such animals as cattle, pigs and sheep (Society for the Study of Reproduction, 38:241 (1988)). In addition, this reference illustrates that the state of the art with respect to generation of transgenic species, other than mice, using modified embryonic stem cells is a well developed methodology.

According to an aspect of the invention there is provided a combined preparation of p53 and ASPP1 and/or ASPP2.

According to a further aspect of the invention there is provided a combined preparation comprising a nucleic acid molecule which encodes a p53 polypeptide, or sequence variant thereof, and at least one nucleic acid molecule which encodes at least one polypeptide, or sequence variant thereof, as represented by the amino acid sequences shown in FIG. 17c and/or FIG. 17d.

In a preferred embodiment of the invention there is provided a nucleic acid molecule which encodes both a p53 polypeptide and at least one polypeptide as represented by the amino acid sequences shown in FIG. 17c and/or FIG. 17d.

In a preferred embodiment of the invention said nucleic acid molecule is part of a vector. Preferably said nucleic acid molecules are operably linked to at least one promoter sequence which controls expression of said nucleic acid molecules. “Tromoter” is an art recognised term and, for the sake of clarity, includes the following features which are provided by example only. Enhancer elements are cis acting nucleic acid sequences often found 5′ to the transcription initiation site of a gene (enhancers can also be found 3′ to a gene sequence or even located in intronic sequences). Enhancers function to increase the rate of transcription of the gene to which the enhancer is linked. Enhancer activity is responsive to trans acting transcription factors which have been shown to bind specifically to enhancer elements.

Promoter elements also include so called TATA box and RNA polymerase initiation selection sequences which function to select a site of transcription initiation. These sequences also bind polypeptides which function, inter alia, to facilitate transcription initiation selection by RNA polymerase.

Adaptations also include the provision of selectable markers and autonomous replication sequences which facilitate the maintenance of said vector in either the eukaryotic cell or prokaryotic host. Vectors which are maintained autonomously are referred to as episomal vectors. Episomal vectors are desirable since these molecules can incorporate large DNA fragments (30-50 kb DNA). Episomal vectors of this type are described in WO98/07876.

A number of viruses are commonly used as vectors for the delivery of exogenous genes. Commonly employed vectors include recombinantly modified enveloped or non-enveloped DNA and RNA viruses, preferably selected from baculoviridiae, parvoviridiae, picomoviridiae, herpesveridiae, poxviridae, adenoviridiae, or picornnaviridiae. Chimeric vectors may also be employed which exploit advantageous elements of each of the parent vector properties (See e.g., Feng, et al.(1997) Nature Biotechnology 15:866-870). Such viral vectors may be wild-type or may be modified by recombinant DNA techniques to be replication deficient, conditionally replicating or replication competent.

Preferred vectors are derived from the adenoviral, adeno-associated viral and retroviral genomes. In the most preferred practice of the invention, the vectors are derived from the human adenovirus genome. Particularly preferred vectors are derived from the human adenovirus serotypes 2 or 5. The replicative capacity of such vectors may be attenuated (to the point of being considered “replication deficient”) by modifications or deletions in the E1a and/or E1b coding regions. Other modifications to the viral genome to achieve particular expression characteristics or permit repeat administration or lower immune response are preferred. Most preferred are human adenoviral type 5 vectors containing a DNA sequence encoding the p53 tumor suppressor gene. In the most preferred practice of the invention as exemplified herein, the vector is replication deficient vector adenoviral vector encoding the p53 tumor suppressor gene A/C/N/53 as described in Gregory, et al., U.S. Pat. No. 5,932,210 issued Aug. 3, 1999 (the entire teaching of which is herein incorporated by reference).

Alternatively, the viral vectors may be conditionally replicating or replication competent. Conditionally replicating viral vectors are used to achieve selective expression in particular cell types while avoiding untoward broad spectrum infection. Examples of conditionally replicating vectors are described in Pennisi, E. (1996) Science 274:342-343; Russell, and S. J. (1994) Eur. J. of Cancer 30A(8):1165-1171. Additional examples of selectively replicating vectors include those vectors wherein an gene essential for replication of the virus is under control of a promoter which is active only in a particular cell type or cell state such that in the absence of expression of such gene, the virus will not replicate. Examples of such vectors are described in Henderson, et al., U.S. Pat. No. 5,698,443 issued Dec. 16, 1997 and Henderson, et al., U.S. Pat. No. 5,871,726 issued Feb. 16, 1999 the entire teachings of which are herein incorporated by reference.

Additionally, the viral genome may be modified to include inducible promoters which achieve replication or expression only under certain conditions. Examples of inducible promoters are known in the scientific literature (See, e.g. Yoshida and Hamada (1997) Biochem. Biophys. Res. Comm. 230:426-430; Iida, et al. (1996) J. Virol. 70(9):6054-6059; Hwang, et al.(1997) J. Virol 71(9):7128-7131; Lee, et al. (1997) Mol. Cell. Biol. 17(9):5097-5105; and Dreher, et al.(1997) J. Biol. Chem 272(46); 29364-29371.

The viruses may also be designed to be selectively replicating viruses. Particularly preferred selectively replicating viruses are described in Ramachandra, et al. PCT International Publication No. WO00/22137, International Application No. PCT/US99/21452 published Apr. 20, 2000 and Howe, J., PCT International Publication No. WO0022136, International Application No. PCT/US99/21451 published Apr. 20, 2000. A particularly preferred selectively replicating recombinant adenovirus is the virus d101/07/309 as more fully described in Howe, J.

It has been demonstrated that viruses which are attenuated for replication are also useful in the therapeutic arena. For example the adenovirus d11520 containing a specific deletion in the E1b55K gene (Barker and Berk (1987) Virology 156: 107) has been used with therapeutic effect in human beings. Such vectors are also described in McCormick (U.S. Pat. No. 5,677,178 issued Oct. 14, 1997) and McCormick, U.S. Pat. No 5,846,945 issued Dec. 8, 1998.

According to a further aspect of the invention there is provided a method to treat a condition which would benefit from an increase in apoptosis comprising administering a preparation comprising a first nucleic acid molecule comprising a nucleic acid sequence which encodes a p53 polypeptide, or sequence variant thereof, and administering a second preparation comprising a second nucleic acid molecule comprising a nucleic acid sequence which encodes a polypeptide, or sequence variant thereof, as represented by the amino acid sequence as shown in FIG. 17c and/or FIG. 17d wherein said preparations are administered simultaneously, sequentially or delayed manner.

According to a further aspect of the invention there is provided a method to treat a condition which would benefit from a stimulation of apoptosis comprising administering a combined preparation according to the invention.

In a preferred method of the invention said condition is cancer.

The preparations of the invention can be typically be administered orally, intravenously, subcutaneously, buccally, rectally, dermally, nasally, tracheally, bronchially, by any other parenteral route, as an oral or nasal spray or via inhalation.

An embodiment of the invention will now be described by example only and with reference to the following Figures:

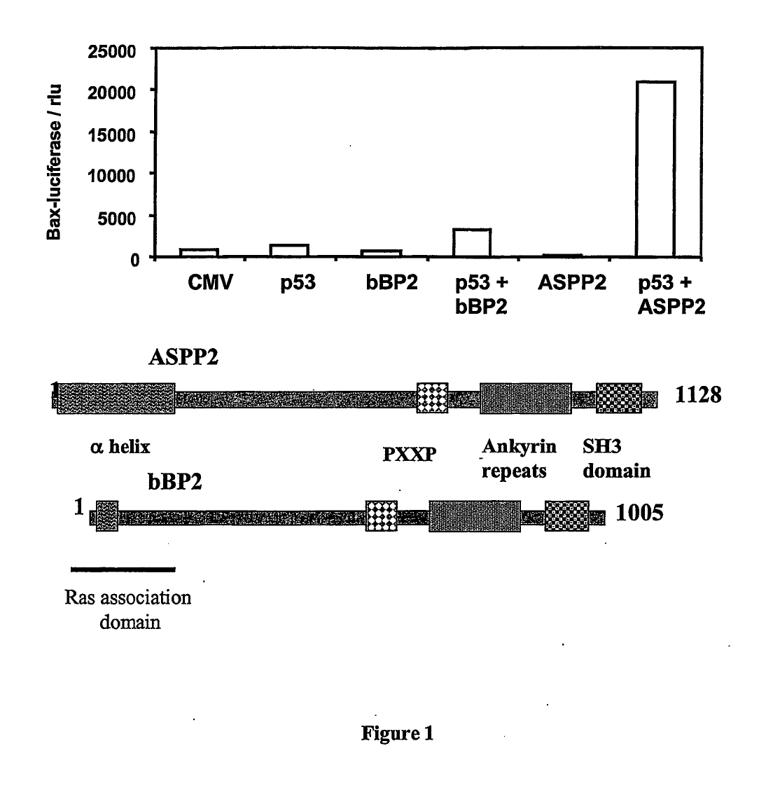

FIG. 1 illustrates the domains within ASPP2 which mediate ASPP2 activity;

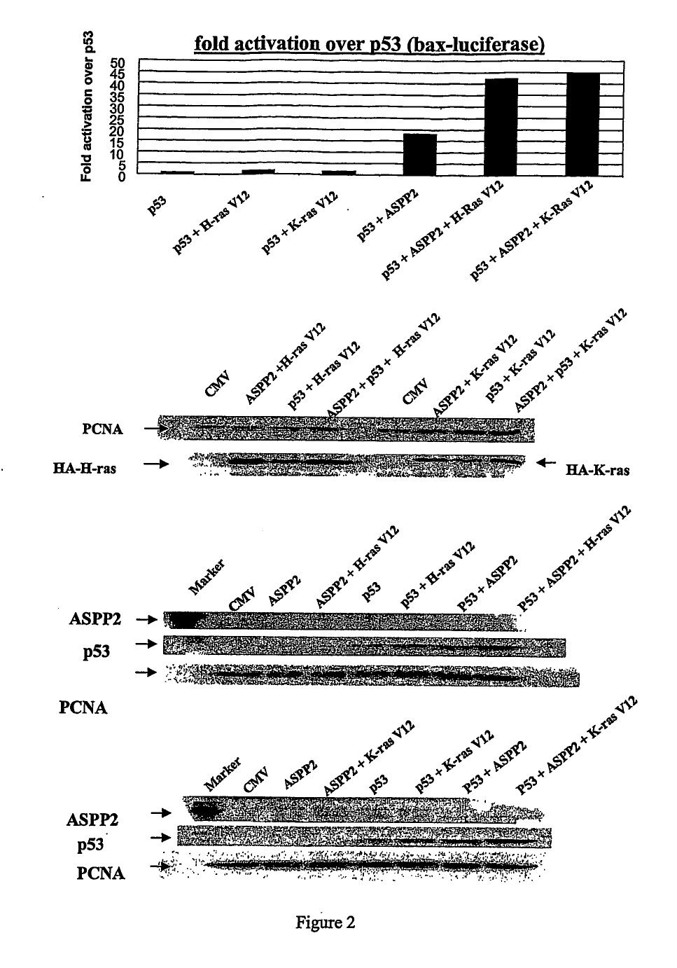

FIG. 2 illustrates that both H-ras V12 and K-ras V12 activate the ASPP2 and p53 synergy. Plasmids expressing CMV, p53, ASPP2 and ASPP2+p53 were transfected in Saos-2 cells either with 1.5 μg of an empty vector, oncogenic H-ras or oncogenic K-ras. All samples were co-transfected with a bax-luciferase reporter. A. A transactivation assay was performed and shown here is the fold activation over p53. The average of duplicate samples are shown. B, C and D. Westem blot of the samples used for transactivation. B. Expression of H-ras V12 and K-ras V12. C. Expression of ASPP2 and p53 for samples co-transfected with H-ras V12. D. Expression of ASPP2 and p53 for samples co-transfected with K-rasV12;

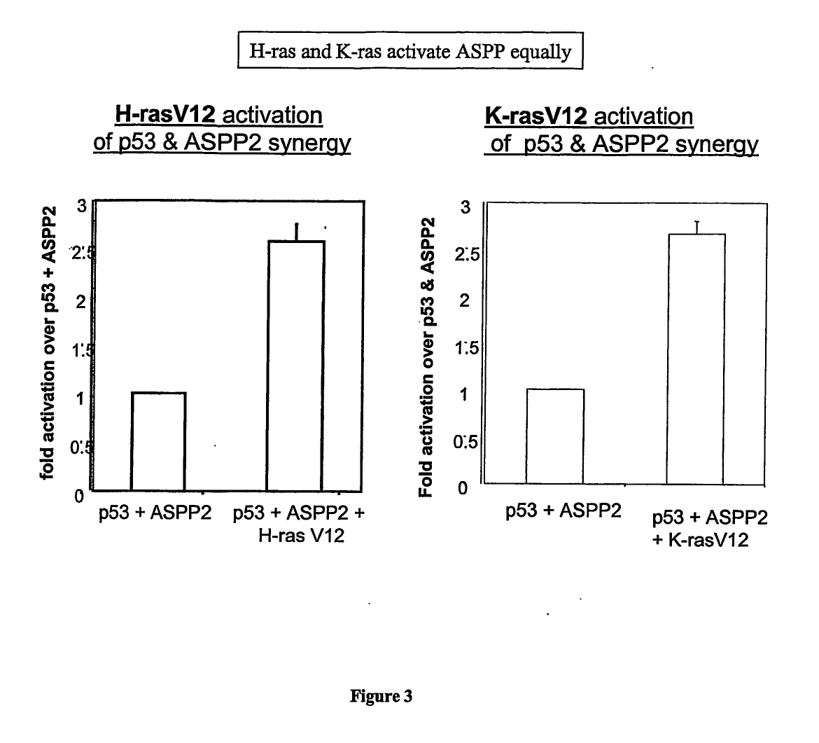

FIG. 3 illustrates that average H-ras V12 and K-ras V12 activation of ASPP2 and p53 synergy;

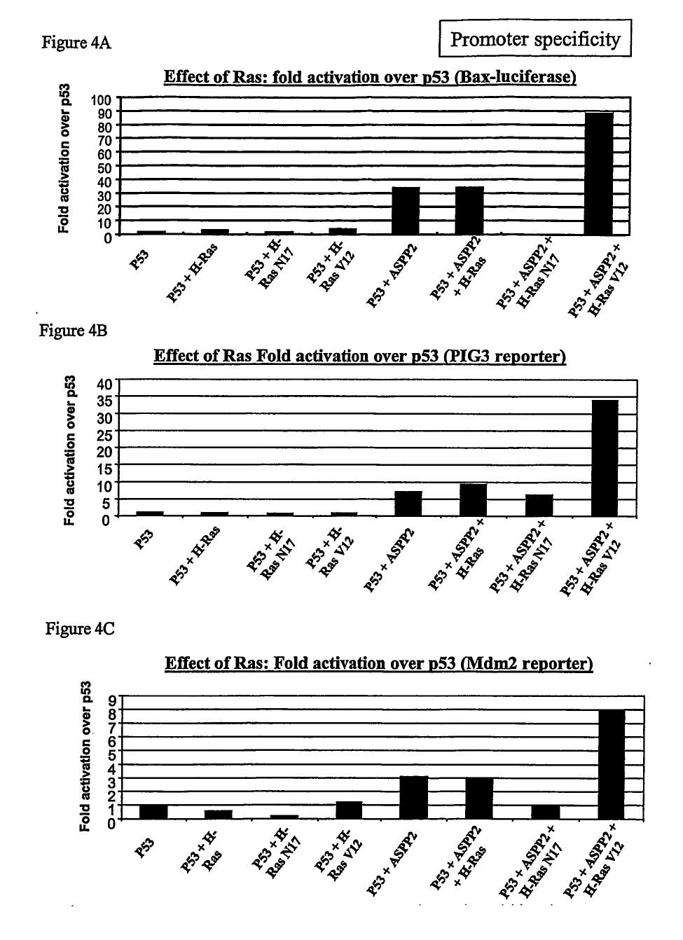

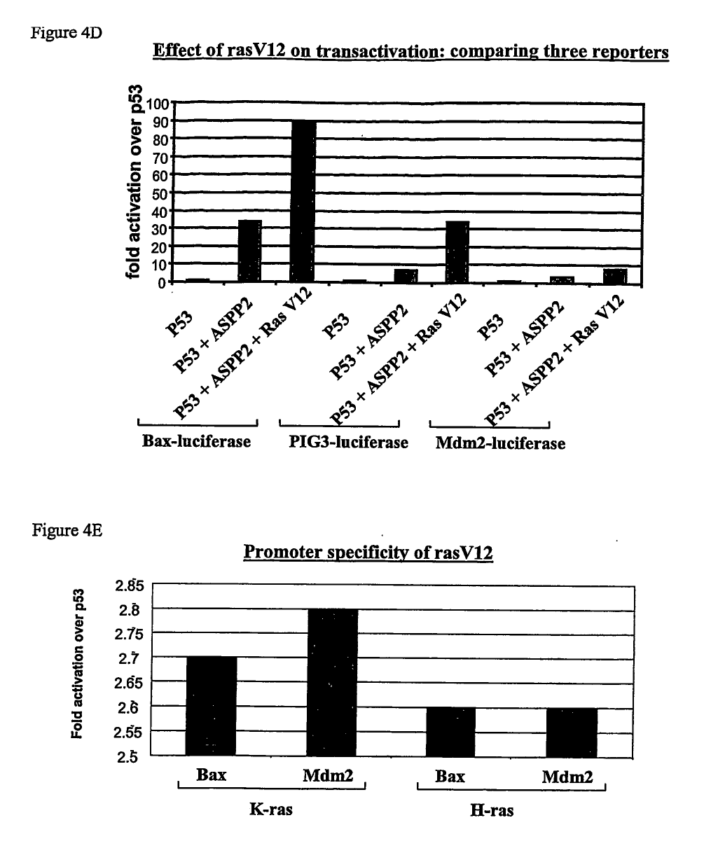

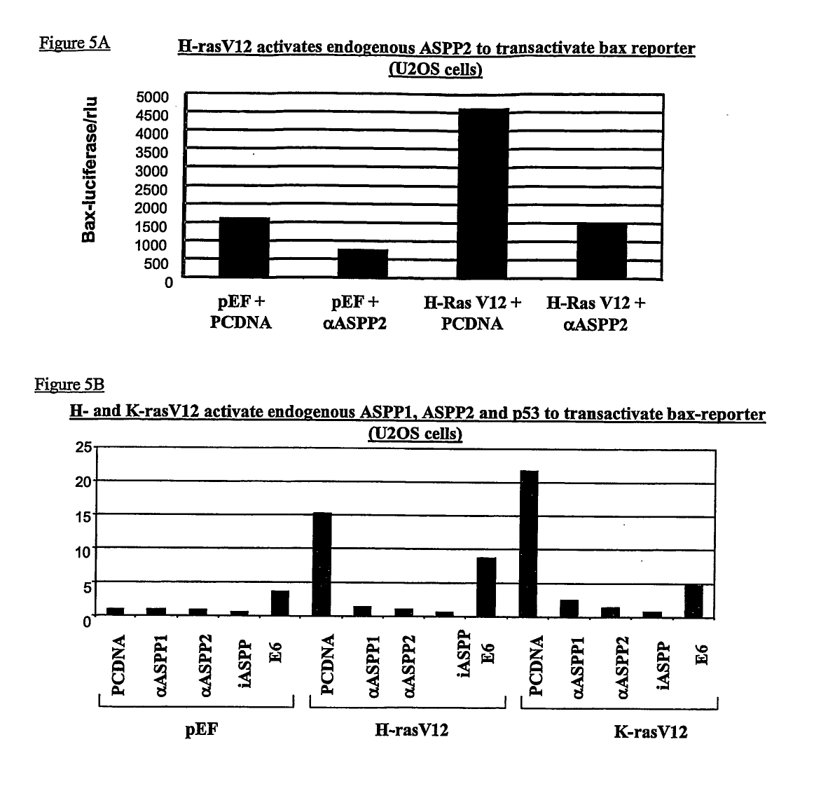

FIG. 4 illustrates Ras and Ras V12 activation of ASPP2 & p53 synergy on different promoters. Plasmids expressing CMV, p53, ASPP2 and ASPP2+p53 were transfected either with 1 μg of an empty vector, wild type H-ras, dominant negative H-ras N17 or oncogenic H-ras V12. All these samples were co-transfected with the indicated reporter. Luciferase activity was determined and the values are in relative luminescent units. The fold activation over p53 is shown. A. Bax-luciferase reporter, B. PIG3-luciferase reporter, C. Mdm2-luciferase reporter. D. The effect of H-rasV12 on ASPP2 and p53 activity on different reporters is shown. E. The fold activation of K-ras V12 and H-rasV12 over p53 and ASPP2 with the Bax-luciferase and the Mdm2-luciferase reporters.;

FIG. 5 illustrates that oncogenic Ras activates endogenous ASPP1/2 and p53 to transactivate Bax-luciferase reporter. A. U2OS cells were transfected with 1 μg of either control vector or H-ras V12, each of which was also transfected with 6 μg of plasmids expressing either PCDNA or anti-sense ASPP2. All samples were co-transfected with bax-luciferase reporter. The lysates were then tested for luciferase levels. The values shown above are averages of duplicates. B. U2OS cells were transfected with 1 μg of a control vector pEF, H-ras V12 or K-ras V12, each of which were then co-transfected with 6 μg of plasmids expressing either PCDNA, anti-sense (α) ASPP1, αASPP2, or E6, or 4 μg of iASPP. All samples were co-transfected with Bax-luciferase. A transactivation assay was done on the lysates. The fold activation above the value of pEF and PCDNA control is shown. All samples were done in duplicate and the mean is shown here;

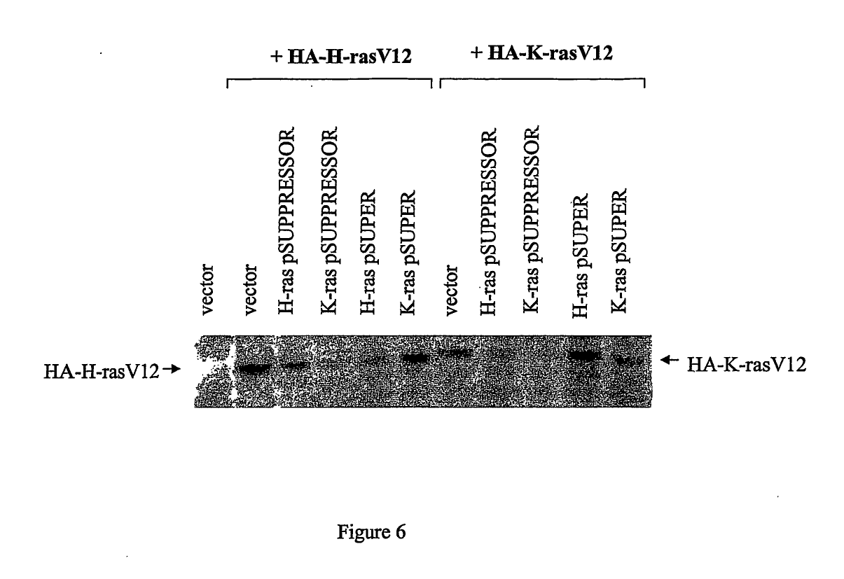

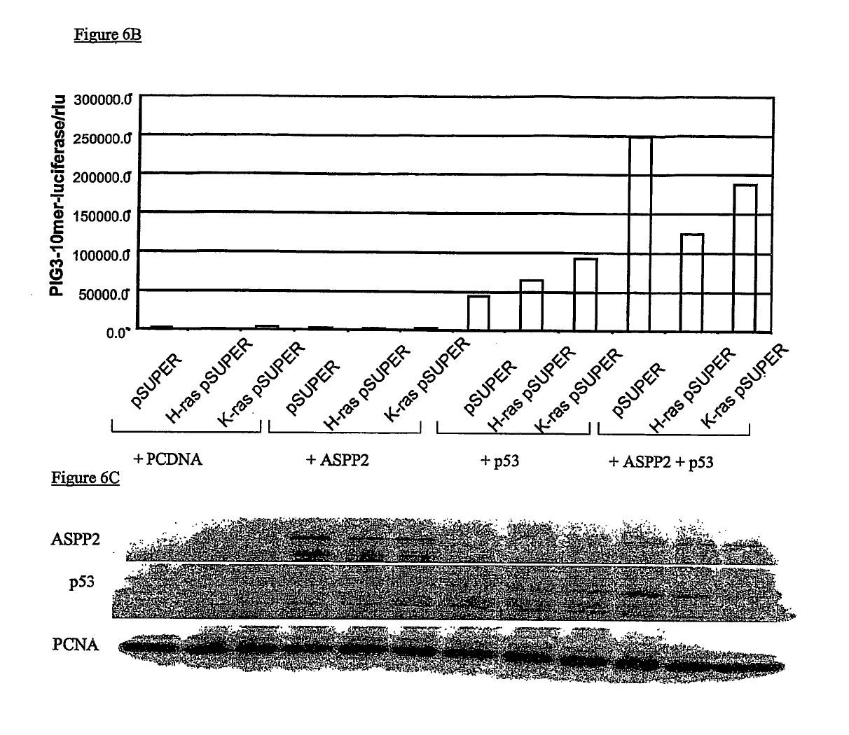

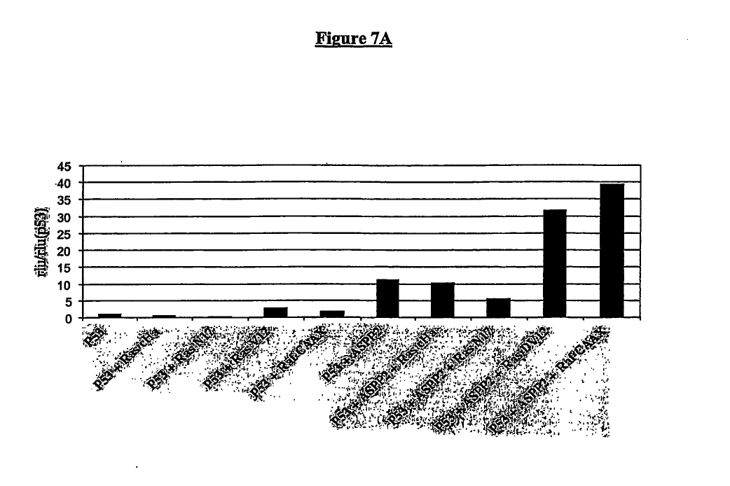

FIG. 6A illustrates the testing of H-ras and K-ras RNAi constructs in pSUPPRESSOR and pSUPER vectors. Both HA-tagged H-rasV12 and HA-tagged K-rasV12 were co-transfected with either H-ras pSUPPRESSOR, K-ras pSUPPRESSOR, H-ras pSUPER or K-ras pSUPER. A western blot was performed with an HA antibody to see whether the different RNAi constructs were able inhibit expression in a specific manner. Effect of Ras RNAi on ASPP2 transactivation activity. FIG. 6B illustrates H-ras pSUPER and K-ras pSUPER co-transfected with ASPP2 and/or p53 and its effect as monitored by the PIG3-luciferase counts. FIG. 6C. A western blot was performed for each sample and blotted for ASPP2 and p53;

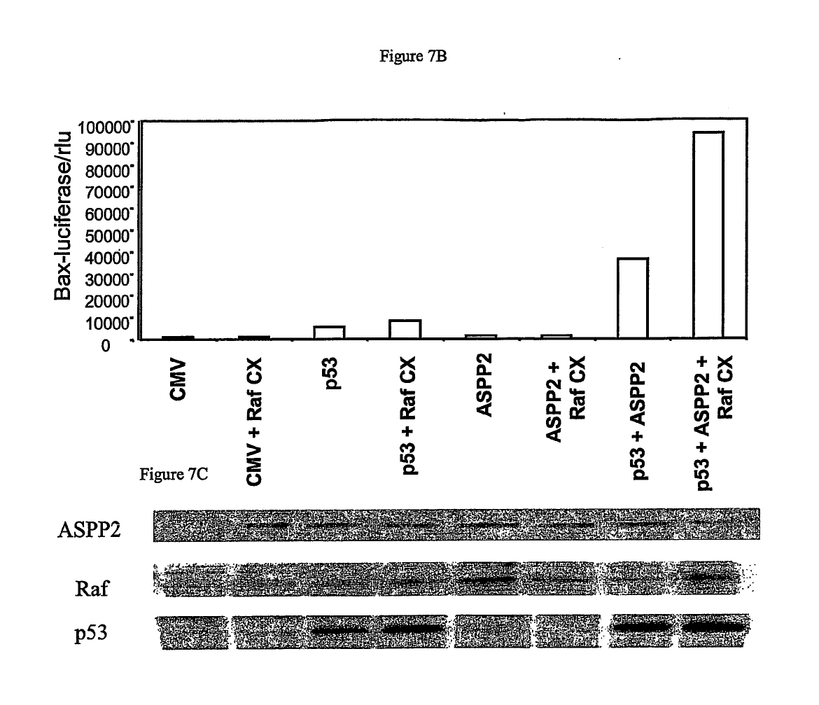

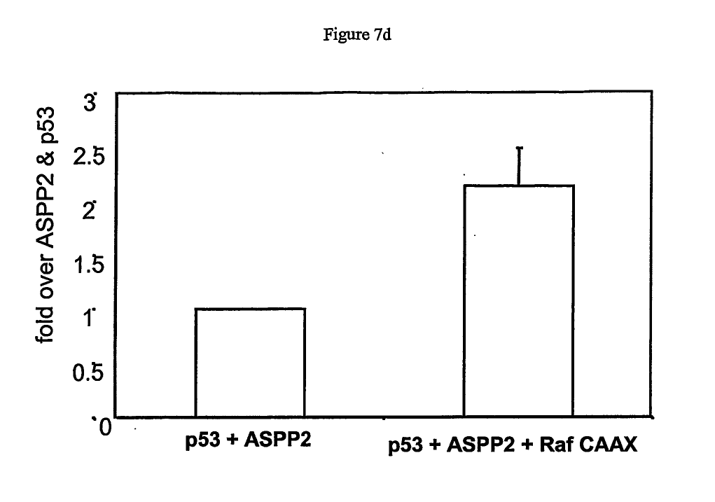

FIG. 7 illustrates that activated Raf increases ASPP2 activity. A. p53 and ASPP2 were co-transfected with wild-type H-ras, dominant negative Ras N17, oncogenic H-ras V12 or Raf CX together with Bax-luciferase reporter. The values shown are the fold increase over p53 alone; FIG. 7B and 7C illustrate activated Raf increases ASPP2 activity. B. p53 and ASPP2 were transfected with or without Raf CX. All samples were co-transfected with bax-luciferase. C. A western blot was performed with the samples shown in 7B and blotted against ASPP2, Raf and p53. FIG. 7D illustrates that activated Raf increases ASPP2 activity. Raf CX increases p53 and ASPP2 synergy 2.5 fold (average of 3 experiments);

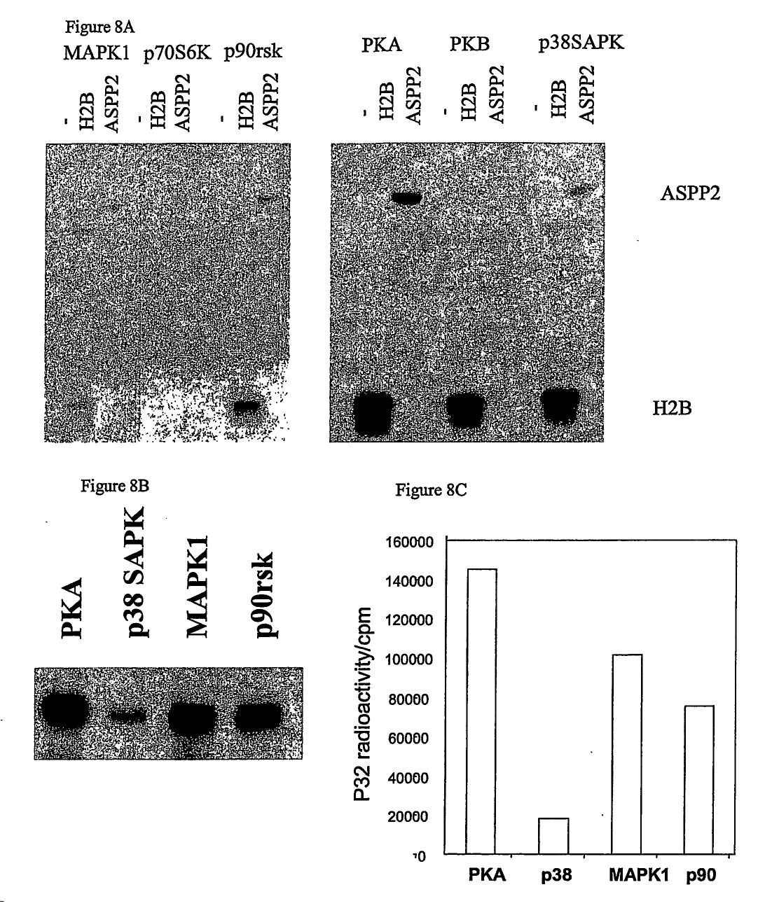

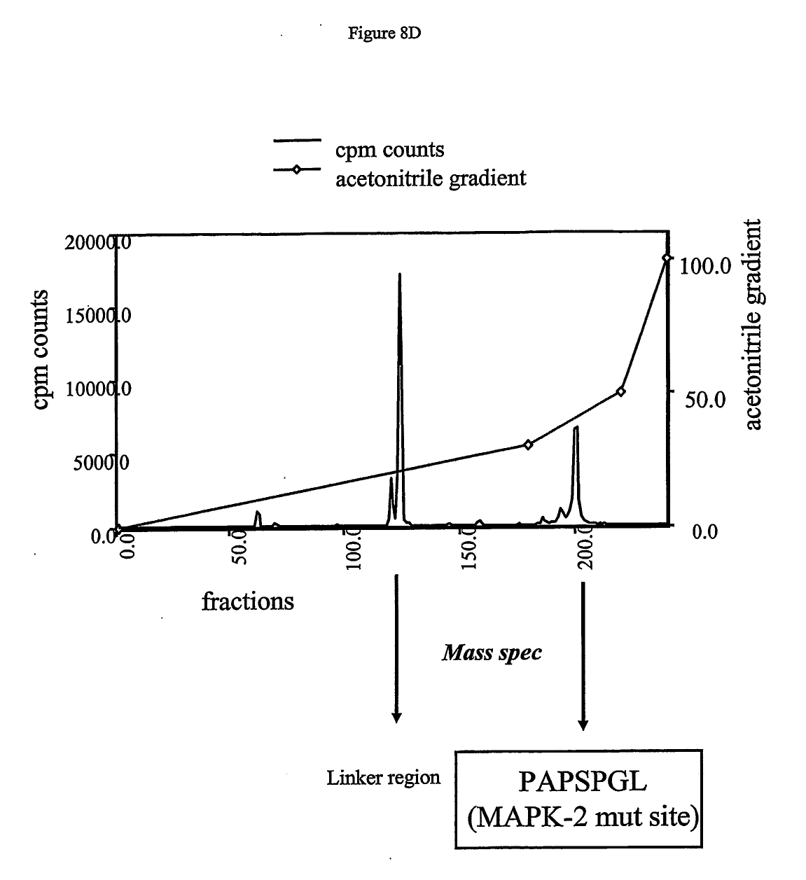

FIG. 8 illustrates in vitro phosphorylation of the C-terminus of ASPP2. A. Small scale screening of ASPP2 phosphorylation by MAPK1, p70S6K, p90rsk, PKA, PKB and p83SAPK. The appropriate enzyme was added to a reaction containing P32 and either H2B as a positive control, ASPP2 or no substrate. B. A large scale in vitro phosphorylation assay was performed with ATP at higher P32 counts. PKA, p38SAPK, MAPK1 and p90rsk were used as kinases. The cpm counts of each band was measure and shown in C. D. The ASPP2 protein phosphorylated by MAPK was trypsinized and put through a High Performance Liquid Chromatography. The radioactive peptides fragments were collected and analysed by mass spectrometry. The second radioactive peak corresponds to a region of ASPP2 containing a putative MAPK phosphorylation site;

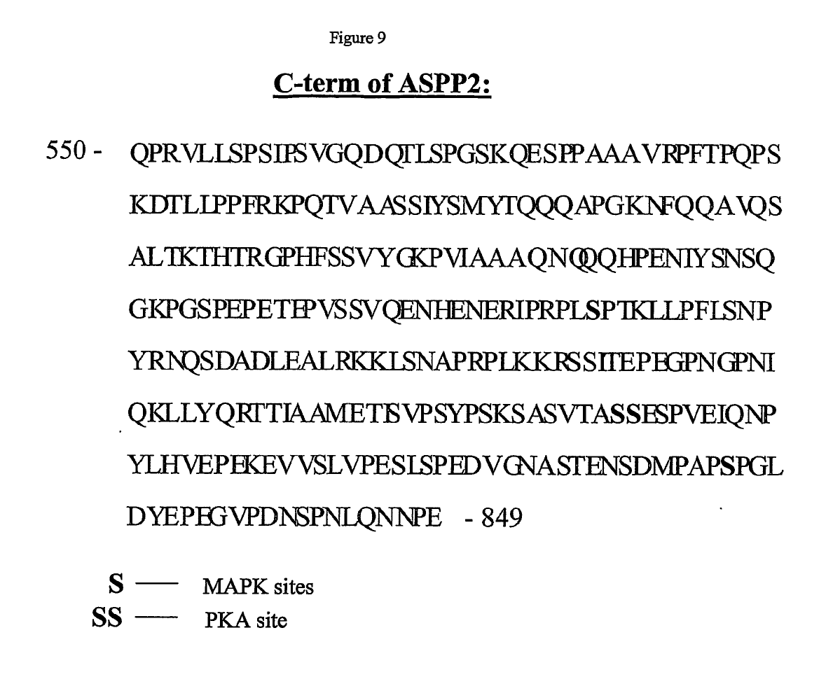

FIG. 9 illustrates ASPP2 phosphorylation mutants. Three ASPP2 point mutant were made by replacing serines by alanine. Mutant 1 has a putative MAPK site mutated (PRPLSPT). Mutant 2 has the two serines at the putative PKA site mutated (TASSESP) and mutant 3 has the in viro phosphorylated MAPK site mutated (PAPSPG);

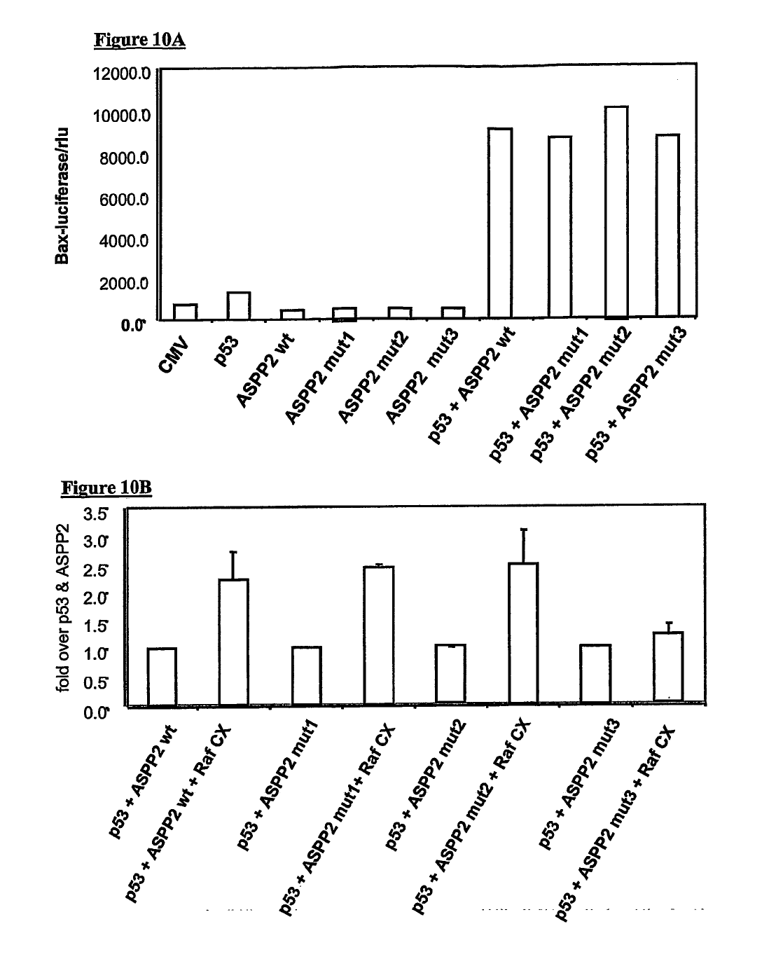

FIG. 10 illustrates that RafCX is not able to transactivate ASPP2 mutant3. A. ASPP2 wild type and mutants were transfected in Saos2 cells with or without p53 and Bax-luciferase reporter activity measured. B. Bax-luciferase, ASPP2 wild type and mutants were transfected with p53 and with or without 1.5 μg of RafCX. The values shown are the fold over ASPP2 and p53. This is the average of 3 experiments;

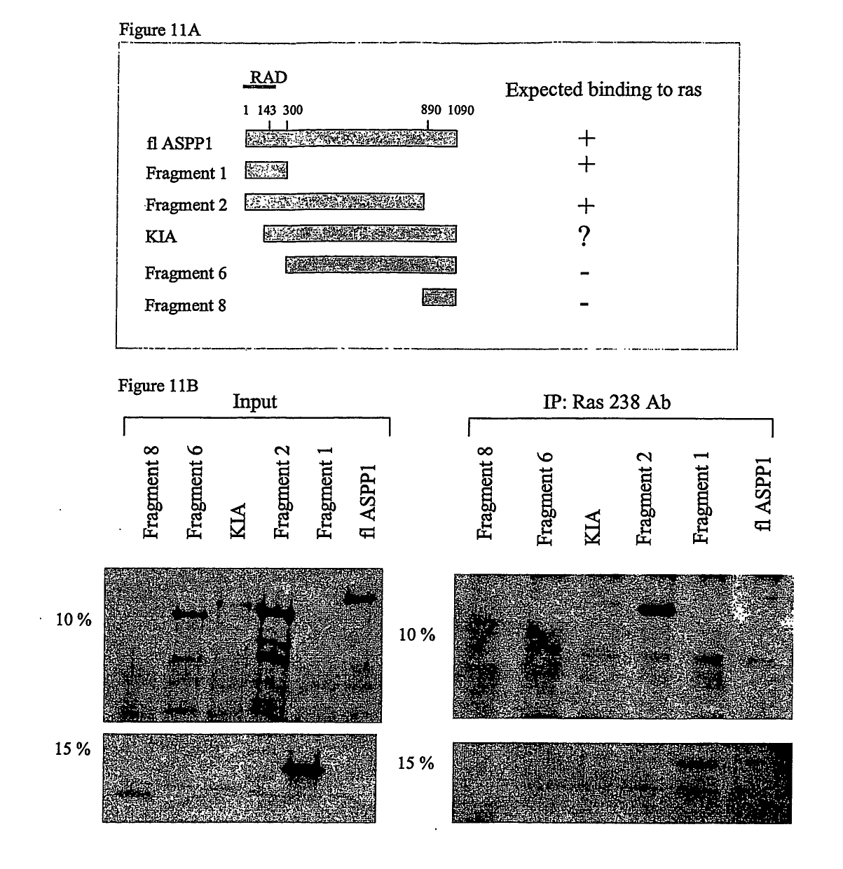

FIG. 11 illustrates that endogenous ras binds the N-terminus of ASPP 1. A. ASPP 1 fragments that either contain or lack the ras-association domain (RAD). The column on the right shows the prediction for the different fragments to bind ras. B. U2OS cells were transfected with 10 μg of the different ASPP1 fragments. Lysates were made and endogenous ras immunoprecipitated with the 238 antibody. The immunoprecipitations were run on a gel and western blotted, probed with V5 to visualize the ASPP1 fragments. The left panel is the input and the right panel the immunoprecipitation;

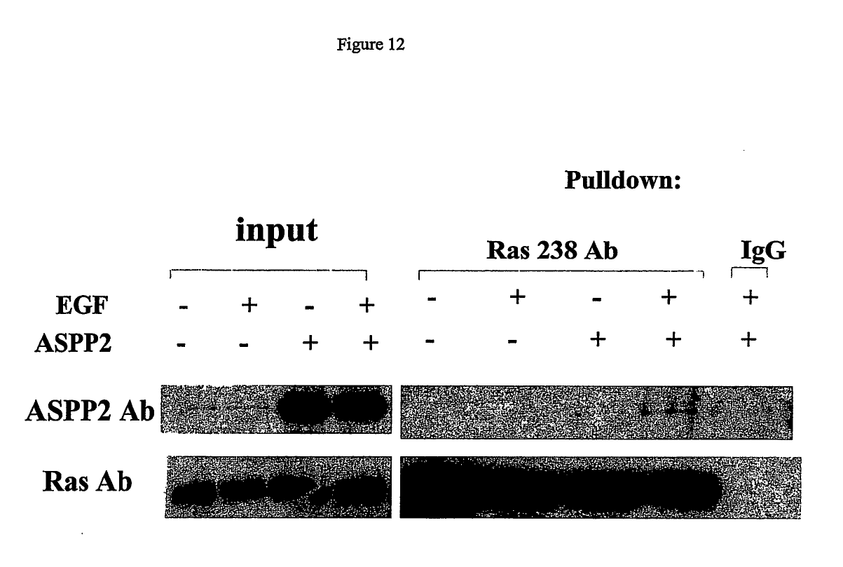

FIG. 12 illustrates that endogenous ras binds ASPP2 after EGF stimulation:. An inducible ASPP2 clone was used for an immunoprecipitation of endogenous ras. The cells were starved overnight, and then either stimulated with EGF or induced for ASPP2 expression or both simultaneously. The left panel shows ASPP2 and ras input whereas the right panel shows the immunoprecipitation of ras (238 antibody) and the immunoprecipitation with the control antibody (IgG);

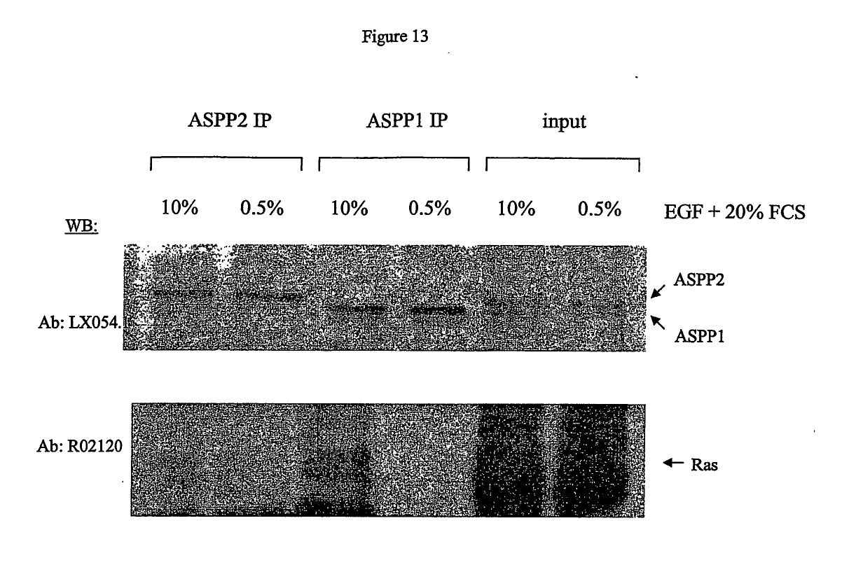

FIG. 13: illustrates that endogenous ASPP1 and ASPP2 bind endogenous activated ras. Saos2 cells were used for an immunoprecipitation of ASPP1 and ASPP2. The cells were starved overnight or grown in EGF and 20% fetal calf serum overnight. The western blot shows that Ras binds ASPP2 slightly more efficiently after EGF and 20% FCS stimulation. Endogenous ras is only co-immunoprecipitated by ASPP1 after the cells have been stimulated with EGF and FCS;

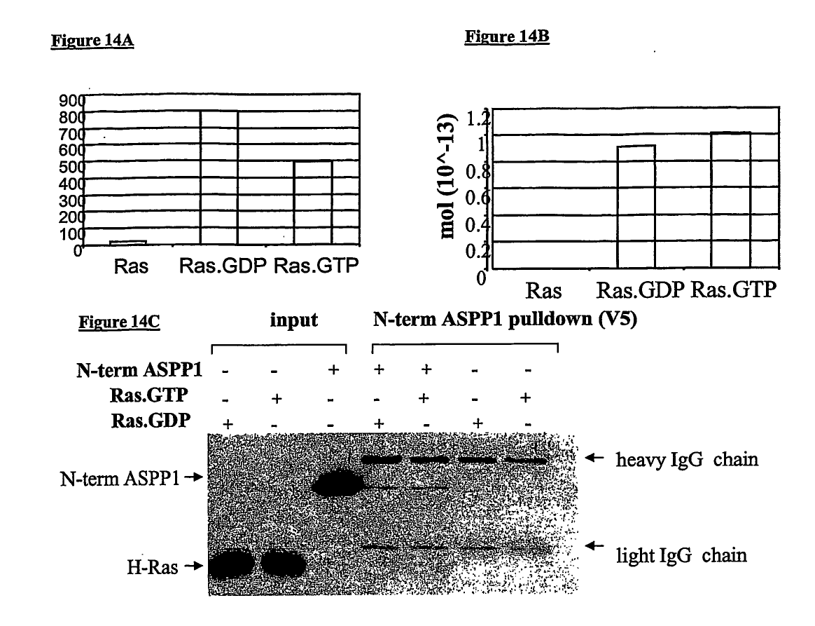

FIG. 14 illustrates that purified N-term ASPP1 binds directly in a preferential manner to Ras.GTP. A. Purified ras was loaded with tritium labelled -GDP, -GTP or not loaded as a control. The cpm counts of the tritium are shown. The differences in cpm is due to the fact that GDP and GTP were not tritium-labelled at the same level. B. Relative loading of ras-GDP and ras-GTP, showing the mol of nucleotide bound to ras. Both ras-GDP and ras-GTP were loaded with an equal amount of nucleotide. C. Loaded ras-GDP and ras-GTP with or without purified N-terminus ASPP1 was immunoprecipitated with a V5 antibody against the ASPP1 fragment. The immunoprecipitations were western blotted against ras to see whether there was direct and preferential binding of ras-GDP and ras-GTP to the amino-terminus of ASPP1. D. The intensity of the ras-GDP and ras-GTP bands pulled down by N-term ASPP1 were measured and shown as a graph. N-terminus ASPP1 binds ras-GTP four fold more strongly than it does to ras-GDP;

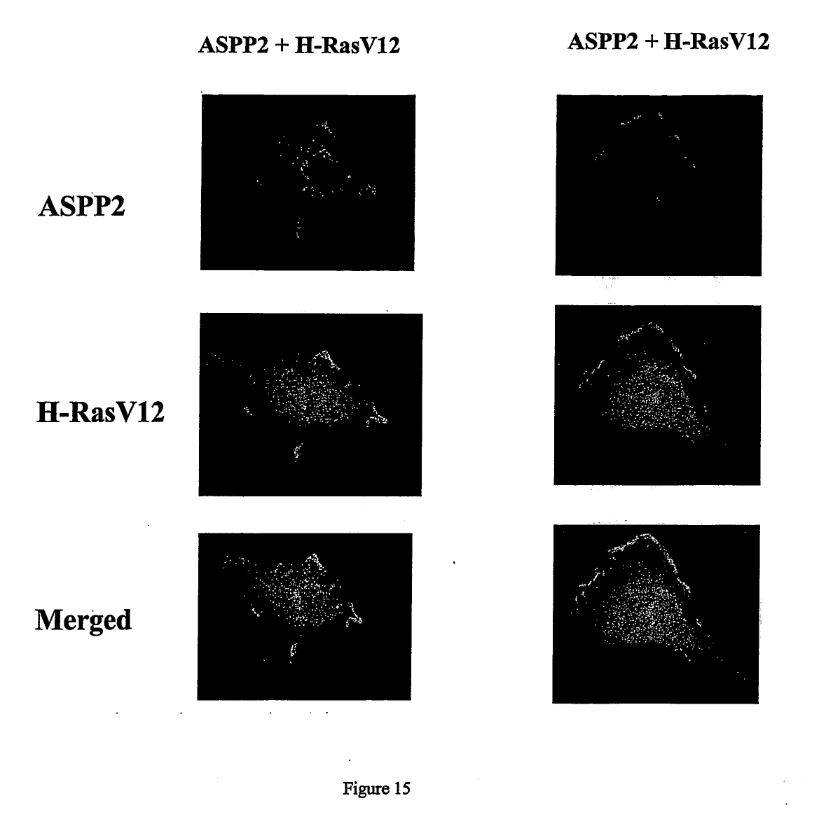

FIG. 15 illustrates that ASPP2 co-localizes with H-ras V12. U2OS cells co-transfected with ASPP2 and H-rasV12 show co-localization of both proteins at the plasma membrane of the cells;

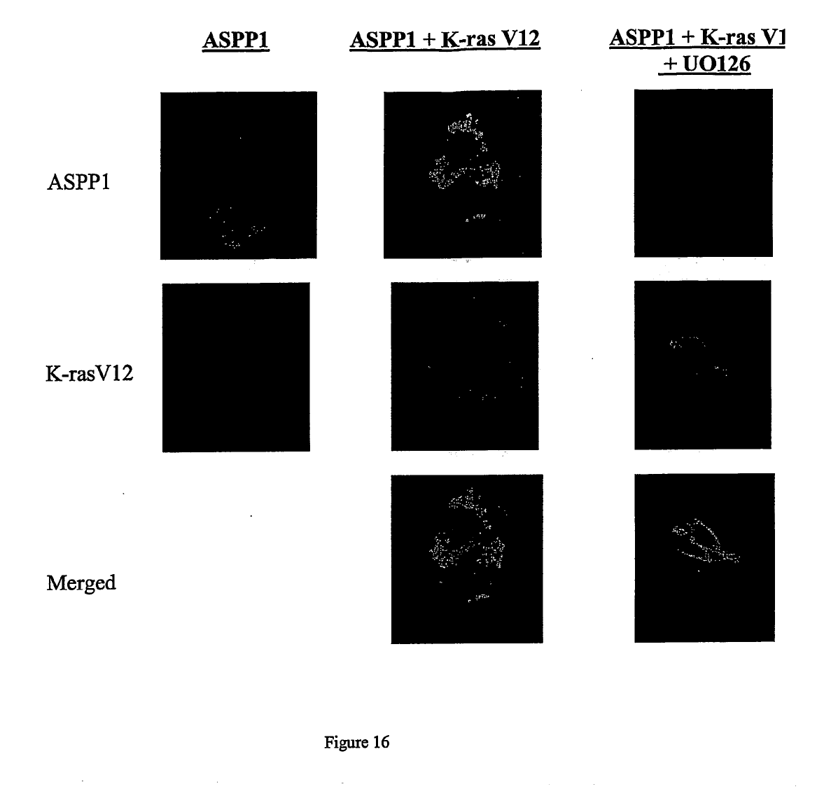

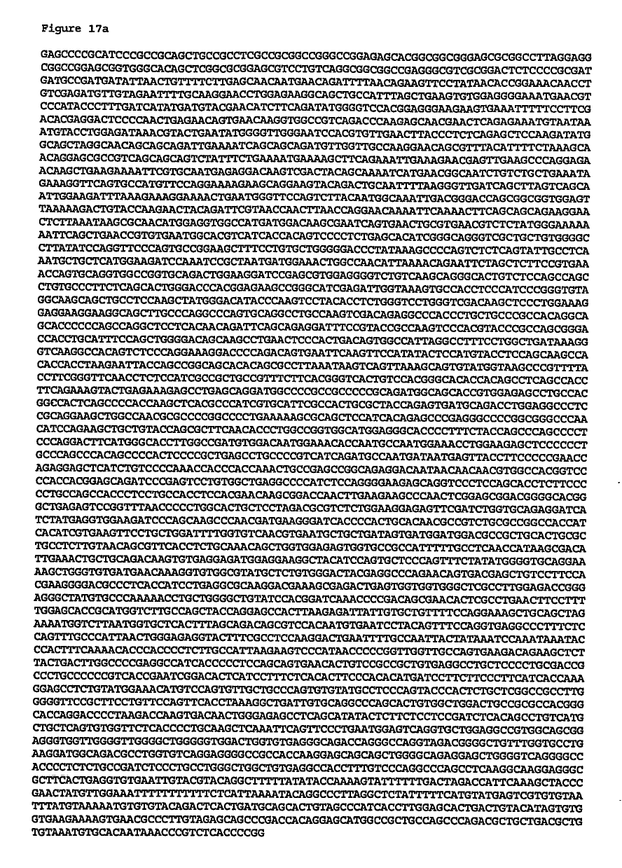

FIG. 16 illustrates that K-rasV12 affects ASPP1 localization in a MAPK-dependent manner. After co-transfection of K-rasV12, ASPP1 changes its sub-cellular localization forming dense doughnut-like shapes. This change in localization is dependent on MAPK as a MAPK inhibitor UO126 reverts this change in localization;

FIG. 17a is the ASPP1 nucleic acid sequence; FIG. 17b is the ASPP2 nucleic acid sequence; FIG. 17c is the ASPP1 protein sequence; FIG. 17d is the ASPP2 protein sequence;

FIG. 18a is the H-Ras wild-type nucleic acid sequence; FIG. 18b is the H-Ras protein sequence; FIG. 18c is the H-Ras oncogenic nucleic acid sequence; FIG. 18d is the H-Ras oncogenic protein sequence; FIG. 18e is the K Ras wild-type nucleic acid sequence; FIG. 18f is the wild-type K-Ras protein sequence; FIG. 18g is the K-Ras oncogenic nucleic acid sequence; and FIG. 18h is the K-Ras oncogenic protein sequence;

FIG. 19a is the MAPK nucleic acid sequence; FIG. 19b is the MAPK protein sequence;

FIG. 20a is the PKA nucleic acid sequence; FIG. 20b is the PKA protein sequence;

FIG. 21a is the phosphatase 1 nucleic acid sequence; FIG. 21b is the phosphatase 1 protein sequence; and

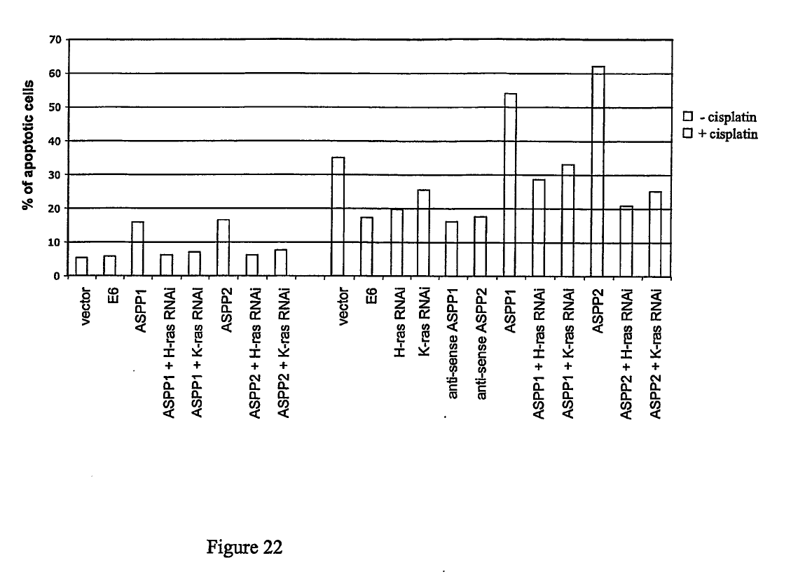

FIG. 22 illustrates the effect of H-ras RNAi and K-ras RNAi on apoptosis.

FIG. 23. Amino-terminus ASPP1 and ASPP2 preferentially bind activated Ras in vitro and in vivo. (A) Sequence homology between the amino-termini of ASPP1 and ASPP2 and the Ras-association domains (RAD) of RGL2 and AF-6. (B) The Ras protein loaded with either tritium-labelled GDP or GTP, was added to V5-tagged recombinant ASPP1 fragment (1-308) and immunoprecipitated with V5 antibody. As a negative control Ras-GDP and Ras-GTP were immunoprecipitated with V5 antibody in the absence of the ASPP1 fragment. The ASPP1 fragment preferentially bound Ras-GTP to Ras-GDP. The intensity of the bands representing immunoprecipitated Ras was quantified and the values shown as a bar graph in the right panel. The standard deviation represents the mean of three independent experiments. (C) ASPP1 fragments (left panel) were transfected in Saos2 cells as indicated and the lysates were immunoprecipitated with either Ras antibody (238) or a control IgG antibody (right panel). (D) Saos2 cells were either starved in 0.5% or stimulated with 20% FCS and 20 ng/ml EGF overnight. Lysates were collected and immunoprecipitated with rabbit anti-ASPP1 or anti-ASPP2 antibodies, ASPP1.88 and ASPP2/77, respectively. The ASPP proteins were detected with mAbASPP1.54.1 that is known to cross-react with both ASPP1 and ASPP2. IP, immunoprecipitation; Ab, antibody; fl, full-length.

FIG. 24 Oncogenic H-Ras and K-Ras enhance the transcriptional activity of p53 via ASPP1 and ASPP2. Saos2 cells were transfected with a bax-luciferase reporter, p53, wild-type or mutant Ras, in the presence of ASPP2 (A and B) or ASPP1 (B), as indicated. The expression of the proteins was verified by Western blot (B). The value of ASPP1+p53 or ASPP2+p53 were taken as 1.0 to reflect the fold increase of ASPP1/ASPP2 and p53 in the presence of mutant Ras (B, right panel). The mean values were derived from three independent experiments;

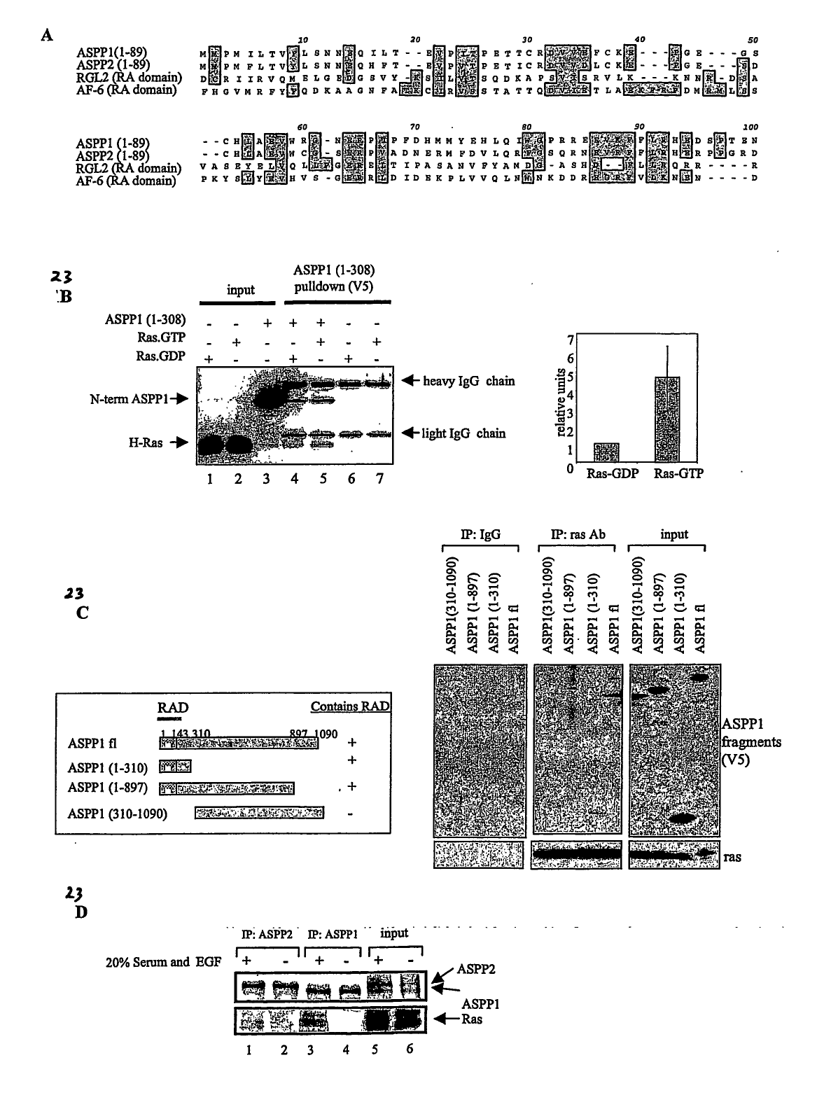

FIG. 25. Endogenous Ras is required for ASPP1 and ASPP2 to stimulated the apoptotic function of p53. (A) Saos2 cells were co-transfected with the p53 reporter PIG3-luciferase, p53, ASPP2, H-Ras RNAi or K-Ras RNAi, as indicated. (B) H-Ras RNAi and K-Ras RNAi were co-transfected with ASPP1 or ASPP2 in U2OS and MCF7 cells. The right panels show the expression levels of p53 and transfected ASPP1 and ASPP2. PCNA was used as a loading control. (C) H-Ras RNAi, K-Ras RNAi, antisense ASPP1, anti-sense ASPP2 and E6 were transfected in U2OS and MCF7 cells as indicated. After transfection, the cells were treated with cisplatin. All cells were cotransfected with a CD20 marker to select transfected cells for FACS analysis;

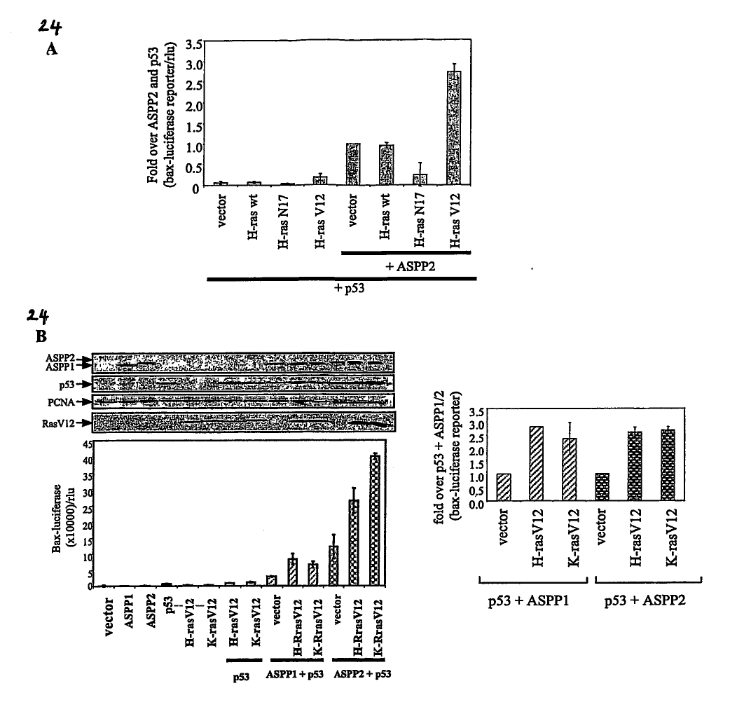

FIG. 26 ASPP2 is phosphorylated by MAPK in vitro and in vivo. (A) Saos2 cells were transfected with bax-luciferase reporter, ASPP2, p53 and RafCX expression plasmids as indicated. The expression levels of ASPP2, Raf and p53 were detected by antibodies DX5410, C-12 and DO-1, respectively (bottom panel). (B) The value of p53 and ASPP2 co-transfected with Bax-luciferase is taken as a value of 1.0 to allow estimation of the effects of RafCX, H-rasV12 and K-rasV12. The mean values are averages of three independent experiments. (C) ASPP1 and ASPP2 contain two putative MAPK phosphorylation sites each in their carboxy-termini at serines 671 and 746, and serines 698 and 827 for ASPP1 and ASPP2, respectively. (D) A carboxy-terminus fragment of ASPP2 (693-1128) containing both putative MAPK phosphorylation site was purified and an in vitro phosphorylation assay performed with recombinant p38 SAPK and MAPK1 in the presence of 32P-labelled ATP (left panel). The intensity of the phosphorylated ASPP2 fragment was measured and represented on a bar graph (middle panel). The MAPK phosphorylated ASPP2 fragment was trypsinized and chromatographed on a Vydac C18 column (right panel). The radioactive peptides were 23 measured by mass spectrometry. The first peak was shown to represent the GST linker region whereas the second represented a region of equal mass to the fragment containing the serine 827. (E) The phosphospecific polyclonal antibody NGH.S4 was raised against a phospho-peptide representing the serine 827 phosphorylation site. An in vitro kinase assay was performed with purified ASPP2 (693-1128), recombinant MAPK and cold ATP. The polyclonal ASPP2/77 antibody recognized the phosphorylated and non-phosphorylated ASPP2 fragments (bottom panel). (F) Saos2 cells were starved for 50 hours and then stimulated with 20% FCS and EGF (20 ng/ml) as indicated. At the indicated times, cells were harvested, lysates prepared and immunoprecipitated with the ASPP2 monoclonal DX54.10 antibody. The top two panels show MAPK phosphorylation status and total MAPK levels in the input. The immunoprecipitates were run on an SDS-PAGE gel and blotted with the phospho-specific NGH.S4 antibody. The blot was reprobed with the ASPP2 polyclonal ASPP2/77 antibody to verify the total amount of ASPP2 immunoprecipitated at each time point (bottom two panels);

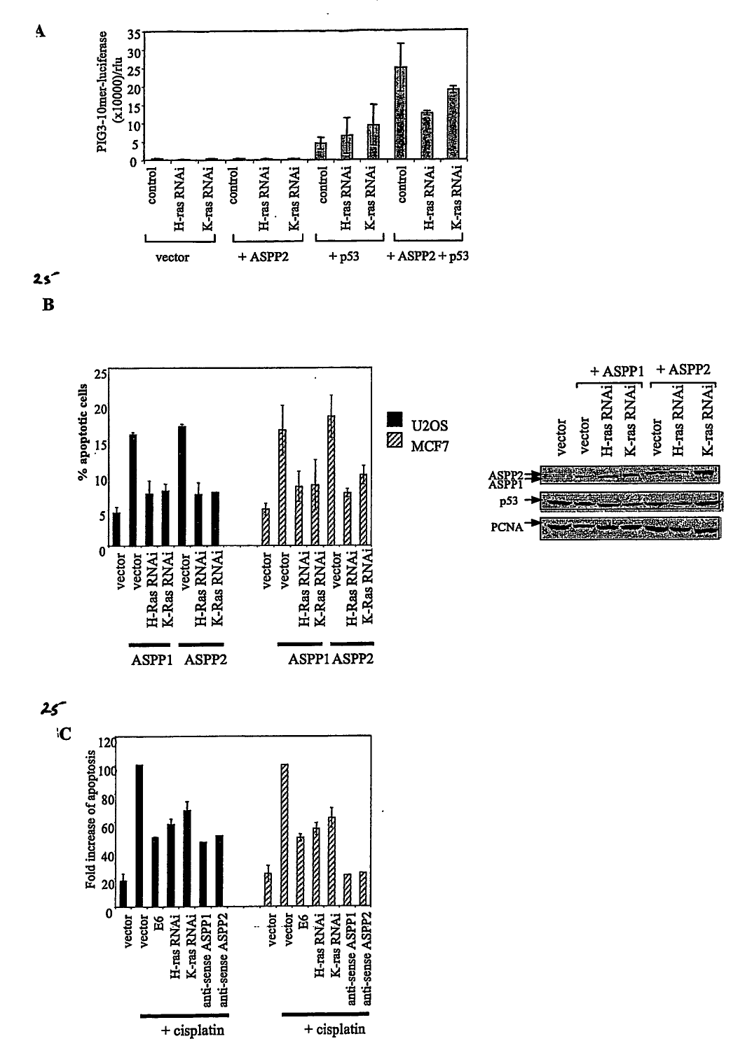

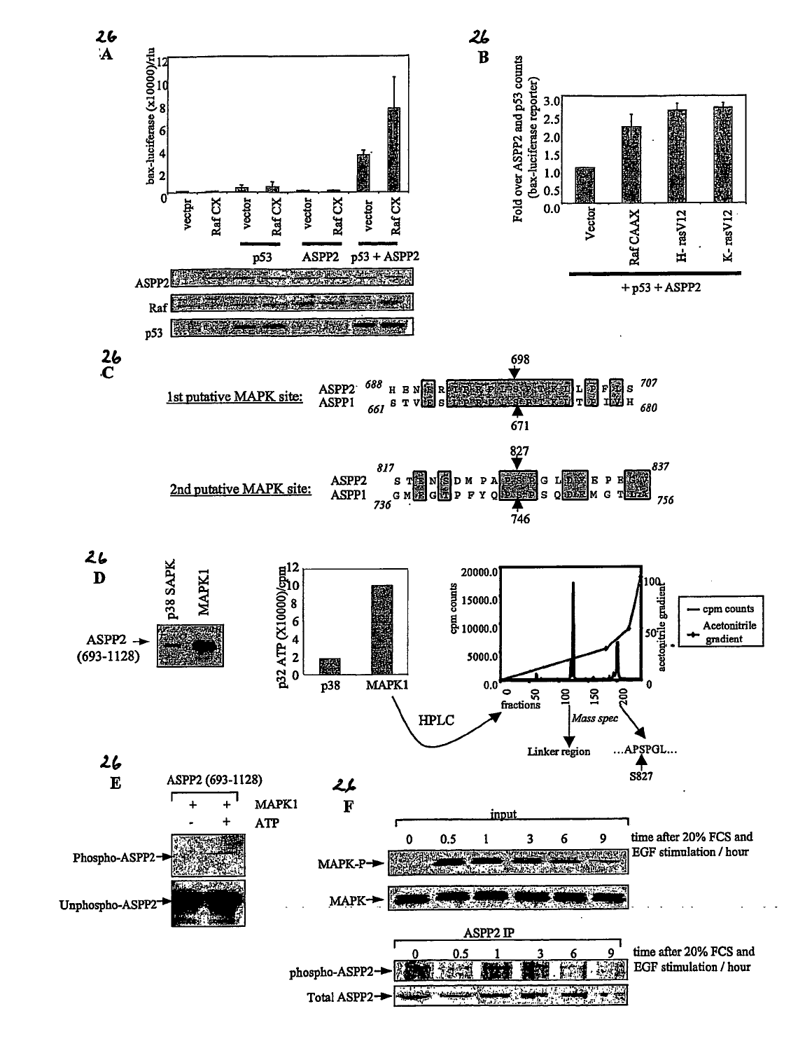

FIG. 27 Endogenous MAPK is necessary for ASPP1 and ASPP2 to stimulate the activity of p53. (A) Saos2 cells were co-transfected with bax-luciferase, ASPP2 and p53. After transfection, medium containing EGF (20 ng/ml) and 20% FCS were added to the cells, in the presence or absence of the MAPK inhibitor UO126 (20 μM). The negative control contained an equal volume of DMSO. The luciferase values are shown in the left panel and the right panel shows the expression levels of the transfected plasmids, endogenous phophorylated MAPK and total MAPK. (B) The putative MAPK phosphorylation sites of ASPP1 and ASPP2 were mutated to alanine, to generate ASPP1 (S671A), ASPP1 (S746), ASPP2 (S698A) and ASPP2 (S827A). Wild-type and mutant ASPP2 (B) or ASPP1 (C) were co-transfected with p53 and Bax-luciferase reporter in the presence or absence of RafCX (B) or K-rasV12 (C) in Saos2 cells, as indicated. Expression levels of ASPP1, p53 and K-rasV12 are shown in the right panel. PCNA was used as a loading control. The graph represents the fold over p53 and ASPP1 or ASPP2 taken from three independent experiments. FCS, foetal calf serum; EGF, epidermal growth factor; and

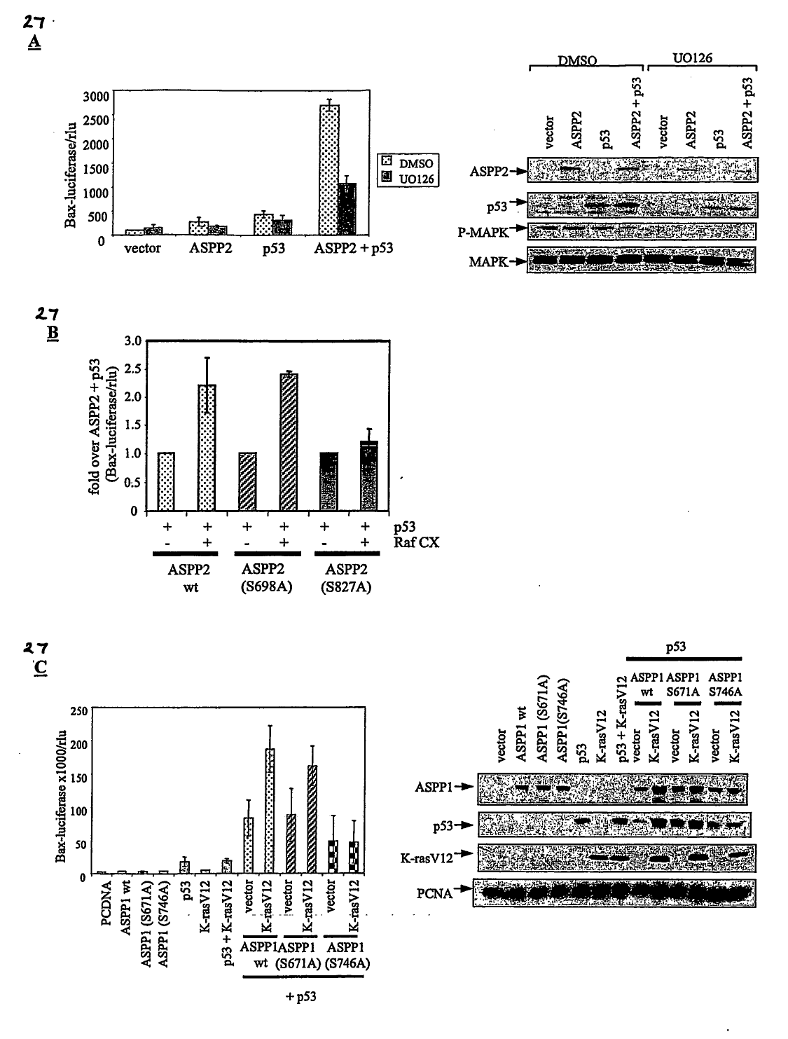

FIG. 28 The Ras-Raf-MAPK pathway stabilizes ASPP1 and ASPP2. Wild-type ASPP2 (A) or mutant ASPP2 (S827A) (B) were transfected in Saos2 cells in the presence or absence of H-rasv12. 16 hours after transfection, the cells were treated with cycloheximide (50 μg/ml) and lysates were made at the times indicated after cycloheximide treatment. The expression of ASPP2 and H-rasV12 were detected with antibodies V5 or HA, respectively. Actin was used as a loading control. (C) MCF7 cells were starved of serum for 24 hours in 0.5% FCS before treatment with 50 μg/ml cycloheximide (CHX) and 20% FCS and EGF, in the presence (right panel) or absence (left panel) of UO126 for the times indicated. 4 mg of lysate was immunoprecipitated with anti-ASPP2 antibody DX54.10 and the presence of ASPP2 was detected by Western blot with the polyclonal ASPP2/77 antibody (top lanes). Phosphorylated MAPK was detected with phospho-specific MAPK antibody and the total MAPK was detected with a MAPK antibody. The intensity of the. ASPP2 protein immunoprecipitated was quantified and the bands at 0 hours of CHX addition was set as 100 for both cells, as shown by the graph (right panel). (D) MDA Panc3 cells were treated with 50 μg/ml cycloheximide, 20 μM UO126 or both cycloheximide and UO126 for the times indicated. The lysate was resolved on SDS-PAGE gels and ASPP1 and ASPP2 protein levels were detected with mAbASPP1.54.2 and DX54.10 antibodies, respectively, and Mcl-1 was detected with an anti-Mcl1 polyclonal antibody.

Methods

Cell culture, Antibodies and Plasmids

Cells were grown in DMEM plus 10% FCS. The mouse monoclonal DO-1 is specific to p53. The V5 epitope is recognized by the mouse monoclonal anti-V5 antibody (Invitrogen) and the haemagglutinin epitope is recognized by the anti-HA antibody (mAb) (Covance). The mouse monoclonal PC-10 is specific to the PCNA protein. CD20Leu is a fluorescein isothiocyanate-conjugated monoclonal antibody specific for the cell surface marker CD20 (Becton Dickinson). The mouse and rabbit antibodies to ASPP1 and ASPP2 were described previously 12 (rabbit anti-ASPP1 polyclonal antibody ASPP1.88, rabbit anti-ASPP2 polyclonal antibody ASPP2/77, mouse monoclonal anti-ASPP2 antibody DX54.10). The mouse monoclonal antibodies mAbASPP1.54.1 and mAbASPP1.54.2 were raised against an amino-terminal fragment of ASPP1 that has high homology to ASPP2. mAbASPP1.54.1 recognizes ASPP1 and cross-reacts with ASPP2 whereas mAbASPP1.54.2 does not cross-react with ASPP2 (data not shown). The mouse monoclonal antibody used against Ras for Western Blots was RO2120 (BD Transduction Laboratories) and to detect Ras in immunoprecipitation the rat monoclonal antibody 238 was used (Santa Cruz). The rabbit polyclonal C-12 (Santa Cruz) was used to detect Raf-1. Mcl-1 was detected by a rabbit polyclonal antibody (Santa Cruz). The deletion mutants of ASPP1 were tagged with the V5-epitope as were full length ASPP1 and ASPP2. H-RasV12 and K-RasV12 were tagged with the haemagglutinin epitope. All expression plasmids used in this study were driven by the cytomegalovirus immediate-early promoter, with the exception of the Ras and RafCX plasmids which were driven by the EF1a promoter. The ASPP1 and ASPP2 mutants were constructed using the QuickChange Site-Directed Mutagenesis Kit (Stratagene). Using the peptide CPAPSpPGLDY (representing residues 824-832) with the serine phosphorylated as an immunogen, a rabbit polyclonal antibody NGH.S4 which specifically recognizes phosphorylated ASPP2 at amino acid 827 was raised. An affinity column was made by cross-linking the phospho-peptide to epoxy-sepharose-6B (Amersham Pharmacia Biotech) according to the manufacturer's instructions. Serum from the final bleed was clarified by centrifugation and filtration through 0.45 μm filter and was supplemented with 1× TTBS (0.5 M NaCl, 20 mM Tris [pH 8.0], 0.1% Tween-20). This was passed over the affinity column and washed with TTBS until the flowthrough had an OD280 nm <0.01. The antibody was then eluted with 0.2M glycine (pH2.8) and neutralized with Tris-HCl (pH 8.0).

RNAi of H-Ras and K-Ras

The mammalian expression vector pSUPER was used for expression of RNAi. In the case of K-Ras RNAi the gene-specific insert specifies a 19-nucleotide sequence corresponding to nucleotides 25-43 downstream of the transcription start site (gttggagctggtggcgtag) of K-Ras, which is separated by a 9-nucleotide noncomplementary spacer (ttcaagaga) from the reverse complement of the same 19-nucleotide sequence. This vector was referred to as K-Ras RNAi. The H-Ras RNAi construct was cloned into the same pSUPER vector with a 20-nucleotide insert corresponding to the nucleotides 299-316 downstream of the transcription start site (tcaaacgggtgaaggactc). These sequences were inserted into the pSUPER.2 backbone after digestion with BglII and HindIII and transformed into TOP10 One ShotTM supercompetent cells (Invitrogen) according to the manufacturer's instructions. Upon ligation, the BglII site is destroyed, allowing for selection of positive clones.

Transactivation Assay

Saos2 cells (7×10⁵) were plated 24 hours prior to transfection in 6-cm-diameter dishes. All transactivation assays contained 1 μg of reporter plasmid. Fifty nanograms of p53, 1 μg of p63© and p73<, 4 μg of ASPP1 or ASPP2 or H-Ras RNAi or K-Ras RNAi, and 1.5 μg of H-rasV12 or K-RasV12 or RafCX expression plasmids were used as indicated. Cells were lysed in reporter lysis buffer 16 to 24 hours after transfection and assayed using the luciferase assay kit (Promega). The fold increase of p53 and ASPP by HRasV12/K-RasV12/RafCX was determined by the activity of p53 and ASPP in combination with RasV12/RafCX divided by the activity of p53 and ASPP alone.

Flow Cytometry

For FACS analysis, 10⁶ cells were plated 24-48 hours prior to transfection in 10-cm diameter plates. All cells were transfected with 2 μg of a plasmid expressing CD20 as a transfection marker, 10 μg ASPP1 or ASPP2, 9 μg H-Ras RNAi or K-Ras RNAi, 6 μg E6, 15 μg anti-sense ASPP1 or anti-sense ASPP2 as indicated. Following transfection, some cells were subsequently treated with 3.5 μg/μl cisplatin for 24 hours. Both attached and floating cells were harvested using 4 mM EDTA/PBS and stained with FITC-conjugated anti-CD20 antibody. The cells were then fixed and stained with propidium iodide. The DNA content of all the cells expressing CD20 was analyzed using a flow cytometry (Becton Dickinson) as described in Hsieh et al.

Protein Biochemistry

For Western blotting, cells were lysed in either NP40 lysis buffer (1% Nonidet P40, 50 mM Tris [pH 8.0], 150 mM NaCl, and 1 mM EDTA), RIPA buffer (150 mM NaCl, 1% Nonidet P40, 0.5% sodium deoxycholate, 0.1% sodium dodecyl sulfate, 50 Mm Tris-HCl [pH 8.0]) or luciferase lysis buffer. Between 35 and 150 μg of extract was mixed with 6× sample buffer and loaded on SDS-PAGE gels. For immunoprecipitation, cells were lysed in Ras IP buffer (20 mM Trish-HCl [pH 7.5], 1 mM EDTA, 1000 mM KCl, 5 mM MgCl₂, 10% v/v glycerol, 1% v/v Triton X-100, 0.05% v/v 2-Mercaptoethanol and protease and phosphatase inhibitors) and the protein concentration determined.

Protein Purification

Both recombinant Ras and the N-terminus of ASPP1 were purified from BL21 bacteria. Protein expression was induced by IPTG (0.2 mM) for 3 hours and the cells were sonicated and then lysed. The lysate was spun at 10000 g and the supernatant recuperated. The recombinant proteins were purified by Glutathionine Sepharose→4B beads (Pharmacia Biotech AB, Sweden) as described by manufacturer in the case of Ras or in the case of N-terminus ASPP1 by nickel columns (Qiagen). The GST tag of Ras was cleaved off with bovine thrombin (Sigma) at a concentration of 5 units/ml and the thrombin removed by P-aminobenzamidine-agarose beads (Sigma). The GST-tagged ASPP2 fragment (693-1128) was purified by Glutathionine Sepharose→4B beads (Pharmacia Biotech AB, Sweden). The protein was concentrated and resupsended in 1× kinase buffer (50 mM Tris-HCl pH 7.5, 270 mM sucrose, 0.1 mM EGTA and 0.1% mercaptoethanol).

Ras GDP/GTP Loading

2.5 μg of recombinant protein purified from Escherichia coli was incubated in a total volume of 320 μl assay buffer containing 2 μCi [3H] GDP or [3H] GTP in a water bath for 10 minutes at 30° C. An aliquot of each sample had its tritium content measured to check the equal loading of GDP and GTP.

In Vitro Immunoprecipitation

1.5 μg of recombinant loaded Ras and 1.5 μg of recombinant ASPP1 fragment (1-308) were mixed together in 500 μl PBS. The lysate was pre-cleared with G-beads for 30 minutes. V5 antibody pre-attached to G-beads was added to the recombinant proteins and incubated for 1 hour at 4° C. The beads were washed three times with NET with 1% NP40 buffer.

In Vivo Immunoprecipitation

1-4 mg of lysate was pre-cleared with protein G beads for 30 minutes at 4° C. and subsequently incubated with antibody pre-bound to protein G beads for 2-16 hours at 4° C. The beads were washed three times with NP40 buffer. The immunoprecipitation beads were mixed with 6× sample buffer and loaded on SDS-PAGE gels. The gels were wet-transferred onto Protran nitrocellulose membrane and the resulting blots were blocked and incubated first in primary antibody and then with the appropriate secondary horseradish peroxidase-conjugated antibody (Dako). The blot was exposed to hyperfilm following the use of enhanced chemiluminescence substrate solution (Amersham Life Science).

In Vitro Phosphorylation Assay.

2 μg of GST-ASPP2 (693-1128) expressed in E. coli was incubated in 30 μl of reaction mixture containing 50 mM Tris-HCl, pH 7.5, 0.1% 2-mercaptoethanol, 1 μM microcystine and 100 μM [©-³²P]ATP (10000 c.p.m./pmol) at 30° C. for 30 minutes. The reactions were terminated as described in Sapkota et al. and analyzed by chromatorgraphy on a C18 column.

Protein Analysis by Cycloheximide Experiments

Cells were either transfected with the appropriate expression plasmids or treated with 20 ng/ml EGF and 20 μM UO126 as indicated, the day prior to cycloheximide treatment. Cycloheximide was added to cells at a final concentration of 50 μg/ml in the appropriate medium. Cells were harvested and lysed at the time points indicated.

### EXAMPLE 1

The Amino Terminus of ASPP1 and ASPP2 is Necessary for its Full Activity

p53 is the most common tumour suppressor protein found mutated in cancers with more than half of all human cancers carrying p53 mutations. p53 is activated by stress signals such as DNA damage and its activation can lead to one of two responses: cell cycle arrest or apoptosis. It has recently been shown that a novel family of tumour suppressor proteins, known as the ASPP family, can interact with p53 and specifically enhance p53-induced apoptosis but not cell-cycle arrest. ASPP1 and ASPP2 enhance the DNA binding and transactivation function of p53 on the promoters of pro-apoptotic genes only in vivo. Mutant ASPP1 and ASPP2 missing the first 150 amino acids are not fully functional, suggesting that the domain is essential for full activity of the ASPP proteins. This amino-terminal domain that is required for ASPP activity contains a putative Ras-association domain (see FIG. 1).

### EXAMPLE 2

Oncogenic H-rasV12 and K-rasV12 Increases ASPP2 Transactivation Activity

It has previously been reported that ASPP2 can increase p53 transactivation on pro-apoptotic genes and we wanted to see whether ras would have any effect on ASPP2 activity. We have shown, using transactivation assays, that both oncogenic H-rasV12 and K-rasV12 can increase ASPP2 activity 2-3 fold (see FIG. 2 and 3) on pro-apoptotic p53 reporters. Knowing that oncogenic RasV12 could activate ASPP2 activity, the roles of wild type Ras and its dominant negative version (Ras N17) were investigated. Interestingly, wild type H-ras seemed to have no effect on ASPP2 and p53 synergy whereas dominant negative ras reduced significantly the synergy, suggesting that endogenous ras activity is needed for the full activity of ASPP2 on the bax-luciferase reporter (FIG. 4A). To see whether the induction of ASPP activity by Ras is promoter specific, different p53 reporters were compared: both pro-apoptotic (such as Bax-luciferase and PIG3-luciferase) and non-apoptotic (Mdm2-luciferase). We can see that in all cases oncogenic ras activates ASPP2 2-3 fold irrespective of the promoter (FIG. 4A-C). Although there does not seem to be any promoter specificity regarding ras activation of ASPP (FIG. 4E), since ASPP2 has an intrinsic pro-apoptotic activity and only activates p53 transactivation on pro-apoptotic reporters, the overall effect of Ras is to significantly increase ASPP2 activity on pro-apoptotic reporters. (FIG. 4D).

### EXAMPLE 3

Oncogenic Ras Can Activate Endogenous ASPP1, ASPP2 and P53 to Transactivate a Pro-apoptotic Reporter

U2OS cells were used to look at the effect of oncogenic ras on endogenous ASPP2 and p53. FIG. 5A shows that H-ras V12 increases the bax-luciferase counts and that this inhibition is inhibited in the presence of anti-sense ASPP2, suggesting that the activity is via endogenous ASPP2. Both ASPP1 and ASPP2 seem necessary for the pro-apoptotic activity of H-rasV12 and K-rasV12. Removal of endogenous ASPP1/2 by anti-sense DNA inhibits oncogenic ras activation to the same levels as adding iASPP (FIG. 5B). The addition of the p53-inhibitor E6 also reduces oncogenic ras activation of bax-luciferase significantly. These results suggest that oncogenic ras can activate bax-luciferase via endogenous ASPP1, ASPP2 and p53.

### EXAMPLE 4

Endogenous H-ras is Necessary for Full ASPP2 Activity

H-ras and K-ras RNAi constructs were made in both the pSUPPPRESSOR and pSUPER vectors. Only the constructs in the pSUPER vector were able to reduce ras levels specifically as shown in FIG. 6A. The pSUPER constructs were therefore tested in a transactivation assay and H-ras RNAi was able to reduce ASPP2 and p53 transcriptional activity as shown in FIG. 6B. All the constructs were expressed (FIG. 6C) although there was no difference in ras levels after H-ras RNAi or K-ras RNAi (figure not shown); this is probably due to the very high endogenous levels of ras in those cells.

### EXAMPLE 5

Ras Activates ASPP2 Via its Raf-MAPK Pathway

Ras is a GTPase which is upstream of many effector pathways, the most well-known of which is the Raf-MAPK pathway. As it was unclear as to whether ras activated ASPP2 directly or via one of its pathways, we looked to see whether an activated form of Raf had any effect on ASPP2 activity. FIG. 7 shows that Raf CAAX (an activated form of Raf) could increase ASPP2 and p53 synergy at least as much as H-rasV12, suggesting that oncogenic ras activates ASPP2 via its Raf pathway.

### EXAMPLE6

The C-term of ASPP2 is Phosphorylated in Vitro by MAPK and Raf CX is Unable to Activate a Phosphorylation Mutant of ASPP2

The purified C-terminus of ASPP2 was used as a substrate to screen for kinases in an in vitro assay. An array of kinases were added to the purified C-terminus of ASPP2 and the phosphorylation status of the protein was analysed using P32 as a phosphorylation marker. MAPK1, PKA, p38SAPK and p90rsk were all found to be able to phosphorylate ASPP2 (FIG. 8A). A larger scale in vitro phosphorylation assay was then performed, using the four enzymes that had screened positive in the first round (FIG. 8B-C). The phosphorylated fragment of ASPP2 was run on a gel, exposed, extracted from the gel and trypsinized. The trypsinized protein was then put through a High Performance Liquid Chromatography (HPLC) with an acetonitrile gradient, the radioactive peptides were then collected and analyzed by mass spectrometry (FIG. 8D). For the MAPK-phosphorylated ASPP2 C-terminus, there were two phosphorylation sites. The first one corresponded to the linker region between the GST and the protein. The second phosphorylated site corresponded to a region of the protein that contains a MAPK-consensus sequence phosphorylation site; PAPSPGL.

ASPP2 phosphorylation mutants were then constructed to see whether this site is phosphorylated in vivo. A serine was replaced by an alanine residue at the MAPK phosphorylation site identified by the in vitro phosphorylation assay (mutant 3), at another MAPK putative site (mutant 1) and at a PKA putative site that was shown to be phosphorylated in vitro (mutant 2) as shown in FIG. 9.

The ASPP2 mutants were compared to wild type ASPP2 in a transactivation assay with p53 using bax-luciferase as a reporter (FIG. 10A). The activity of all three ASPP2 mutants were equivalent to ASPP2 wild type activity. We then wanted to see if RafCX was able to increase ASPP2 wild type and mutants activity. p53 and ASPP2 wild type or mutants were transfected with and without RafCX and the bax-luciferase reporter activity measured. The values shown in FIG. 10B show the fold of activation of RafCX over p53 and ASPP2 values. RafCX is shown to increase ASPP2 wild type, mutant 1 and mutant 2 activity but not ASPP2 mutant 3 activity. ASPP2 mutant three is mutated in the putative MAPK site that was shown to phosphorylate ASPP2 in vitro. This suggests that RafCX, by activating its downstream effector pathway MAPKK-MAPK, leads to MAPK phosphorylation of ASPP2 at the serine site PAPSPGL (amino acid 827) and that phosphorylation on this serine is necessary for RafCX activation of ASPP2.

### EXAMPLE 8

H-ras Binds to ASPP1 in its Amino-terminus

As ASPP1 and ASPP2 contain a putative ras-association domain in their amino-terminus, we investigated whether ASPP was able to bind ras. We used different fragments of ASPP1 to see what parts of the protein bound to ras (FIG. 11A). Fragment 1 contains the N-terminus of the protein, fragment 2 lacks the C-terminus, KIA lacks part of the ras-association domain, fragment 6 lacks the entire ras-association domain and fragment 8 contains only the carboxy-terminus. Endogenous ras was immunoprecipitated and the fragments blotted for by western blot. As FIG. 11B shows, all fragments containing the putative ras-association domain (full length ASPP1, fragment 1 and fragment 2) were pulled down by endogenous ras. The fragments lacling the ras-association domain (fragment 6 and fragment 8) were unable to bind to endogenous ras. Fragment KIA which contained part of the ras association domain was immunoprecipitated by endogenous ras; this might be due to this fragment heterodimerizing with fill-length, wild-type endogenous ASPP1 which contains the entire ras-association domain.

### EXAMPLE 9

Activated Endogenous Ras Binds ASPP1 and ASPP2

EGF ligand activates EGFR, which leads to the recruitment of a guanine nucleotide exchange factor (GNEF). Once recruited to the plasma membrane and in close proximity to Ras, the GNEF leads to the exchange of GDP- to GTP-associated ras, making ras active and able to activate its downstream effectors. Thus EGF is able to activate endogenous ras in a physiological manner. An ASPP2 inducible clone was used to see whether endogenous, wild-type ras bound ASPP2 with different activity after stimulation with EGF. An immunoprecipitation was performed with endogenous ras, with and without ASPP2 and ras induction. FIG. 12 shows that there is very little background binding of endogenous ras and endogenous ASPP2 after cells have been starved for 24 hours. Inducing ASPP2 expression shows a small band of ASPP2 being co-immunoprecipitated by endogenous ras. Inducing endogenous ras by EGF and foetal calf serum leads to a significant increase in ras and ASPP2 binding.

An immunoprecipitation was performed to determine whether endogenous ras is able to bind to endogenous ASPP1 and ASPP2. FIG. 13 shows that induced ras is co-immunoprecipitated with endogenous ASPP1, whereas non-induced ras was not able to bind to ASPP1. Although the difference in binding of ASPP2 to induced versus non-induced ras was not as clear, we can still see a slight increase of ASPP2 binding to induced ras compared to non-induced ras.

### EXAMPLE10

The Amino-terminus of ASPP1 Binds ras.GTP More Efficiently than ras.GDP

In order to determine whether the interaction of ras and ASPP1/2 is direct, as we would expect if ASPP contains an active ras-association domain, we performed an in vitro binding assay with purified ras and amino-terminus of ASPP1. As suggested with the EGF-dependent binding of ras to ASPP, we speculated that ras in its GTP form could bind the amino-terminus of ASPP1 more efficiently than ras in its GDP form. We therefore purified ras and loaded it with either GDP or GTP (FIG. 14A-B). The loaded ras was mixed with the purified amino-terminus-ASPP1 or without it as a control and the amino-terminus of ASPP1 was immunoprecipitated with a V5 antibody. As FIG. 14C-D shows, the N-terminus of ASPP1 binds ras-GTP with four-fold more efficiency than to ras-GDP.

### EXAMPLE 11

Oncogenic ras Co-localizes with ASPP2 But not ASPP1 and Changes the ASPP1 Cellular Localization.

U2OS cells co-transfected with oncogenic H-rasV12 and ASPP2 clearly show co-localization at the plasma membrane of the cells (FIG. 15). However H-rasV12 and ASPP1 are not seen to co-localize. ASPP1, unlike ASPP2, is not found at the plasma membrane (FIG. 16). After co-transfection of K-rasV12, ASPP1 forms dense doughnut-like shapes in the middle of the cells. However, after addition of a MAPK-inhibitor UO126, ASPP1 resumes its “normal” cellular localization pattern.

### EXAMPLE 12

ASPP1 and ASPP2 Interact with Activated Ras in Vitro and in Vivo

Residues 1-89 of ASPP1 and ASPP2 share sequence homology with the Ras association domain of RBL2 and AF6, suggesting that ASPP1 and ASPP2 may bind Ras (FIG. 23A)

The ability of ASPP1 to interact with Ras was confirmed by the observation that the purified ASPP1 fragment (1-308), containing the Ras-association domain (RAD), interacted with purified Ras in vitro. The ASPP1 amino-terminus bound Ras-GTP with a 4-fold higher affinity than Ras-GDP (FIG. 23B, compare lanes 4 and 5). To illustrate that only the amino-terminal region of ASPP1 binds Ras in vivo, various ASPP1 truncation mutants were constructed. All truncation mutants of ASPP1 containing the putative RAD were complexed with endogenous Ras in vivo except for ASPP1 (310-1090) which lacked the RAD (FIG. 23C). To provide in vivo evidence that ASPP1 and ASPP2 preferentially interact with active Ras (Ras-GTP), Saos2 cells were starved for 20 hours and then stimulated with 20% foetal calf serum (FCS) and epidermal growth factor (EGF) to enrich for Ras-GTP.

More Ras was detected in the ASPP1 and ASPP2 immunoprecipates derived from serum- and EGF-stimulated cell lysates than in those unstimulated (FIG. 23D, compare lanes 1 to 2 and 3 to 4 for ASPP2 and ASPP1, respectively). Hence, endogenous ASPP1 and ASPP2 have higher binding affinity to Ras in cells stimulated with serum and EGF than in non-stimulated cells.

### EXAMPLE 13

ASPP1 and ASPP2 have been shown to synergize with p53 to induce the transcription of pro-apoptotic genes and this activity requires intact ASPP1 and ASPP1/2. Therefore, to test the biological significance of the Ras/ASPP interactions, oncogenic H-RasV12, which is constitutively in its GTP-bound form, was co-transfected with ASPP2 and p53 in Saos2 cells as indicated. The results shown in FIG. 24A illustrated that H-RasV12 had a small stimulatory effect on the transactivation function of p53 alone. In the presence of ASPP2, however, the expression of H-RasV12 stimulated the transactivation function of p53 by approximately 3-fold (FIG. 24A). Interestingly, when a dominant negative form of H-Ras, known as H-RasN17, was co-transfected with ASPP2 and p53, the transactivation activity of p53 was inhibited. This inhibition was via ASPP2 since H-RasN17 transfected alone with p53 did not have such a strong inhibitory effect: H-RasN17 inhibited p53 and ASPP2 activity more than 4 fold whereas it inhibited p53 via endogenous ASPP about 1.5 fold.

In contrast, wild-type Ras had very little effect on the transactivation function of p53. The levels of Ras bound to GTP or GDP is tightly regulated in cells and increasing the amount of total Ras by transfection does not necessarily lead to increased levels of Ras-GTP in cells, which could explain why wild-type H-Ras had no effect on ASPP2 activity. Both H-RasV12 and K-RasV12 could stimulate the transactivation function of p53 to the same extent, namely 2.5 fold, and this was mediated by ASPP1 and ASPP2 (FIG. 24B). This increase was not caused by an increase in p53 expression (FIG. 24B).

### EXAMPLE 14

Endogenous Ras is Required for the Full Activity of ASPP1 and ASPP2

The requirement of endogenous Ras to stimulate the activity of p53 via ASPP1 and ASPP2 was tested using RNA interference (RNAi). H-Ras RNAi and K-Ras RNAi were cloned into a PSUPER vector and their ability to specifically reduce the expression of H-Ras or K-Ras was confirmed (data not shown). The results in FIG. 25A show that removing endogenous H-Ras or K-Ras by RNAi reduced the ability of ASPP2 to stimulate the transactivation function of p53 on the promoters of the pro-apoptotic gene PIG3, the same was true for the Bax-luciferase reporter (data not shown). Similarly, reduced expression of endogenous H-Ras or K-Ras mediated by RNAi also significantly inhibited the percentage of apoptotic cells induced by the ASPP1 and ASPP2 expression 6 in two p53 wild-type expressing tumour cell lines, U2OS and MCF7 (FIG. 25B). Furthermore, the percentage of apoptotic cells induced by the commonly used chemotherapy drug, cisplatin, was reduced following the inhibition of H-Ras or K-Ras expression by RNAi (FIG. 25C). At least part of the cisplatin-induced apoptosis was dependent on the expression of p53 and ASPP1 or ASPP2 since the expression of the human papilloma viral protein E6 or the presence of ASPP1 or ASPP2 anti-sense RNA reduced the percentage of apoptosis in both U2OS and MCF7 cells (FIG. 25C).

All these results demonstrated that endogenous H-Ras and K-Ras are necessary for the full activities of ASPP1 and ASPP2.

### EXAMPLE 15

Ser827 of ASPP2 is Phosphorylated by MAPK in Vitro and in Vivo

One of the most studied downstream pathways of Ras signalling is the Raf-MAPK pathway which acts as a phosphorylation cascade. To investigate whether the Ras-Raf-MAPK pathway stimulates p53 via ASPP1 and ASPP2, an activated form of Raf known as Raf CX, was used in a transactivation assay with a bax-luciferase reporter (FIG. 26A). FIG. 26B showed that Raf CX was able to stimulate the transactivation function of p53 via ASPP2 to the same extent as oncogenic Ras. This suggested that Ras might stimulate the activity of ASPP1 and ASPP2 via the Raf-MAPK pathway. Raf is known to activate MEK which, once activated, can phosphorylate MAPK1 and MAPK2 (also known as Erk1 and Erk2). MAPK1/2 can itself phosphorylate many substrates and ultimately lead to cell differentiation, cell growth or apoptosis. We have found two putative MAPK phosphorylation sites in ASPP1 and ASPP2, both situated in their carboxy-terminus. The first site is at amino acids 671 and 698 for ASPP1 and ASPP2, respectively, and the second putative phosphorylation site is at amino acids 746 and 827 for ASPP1 and ASPP2, respectively (FIG. 26C). An in vitro phosphorylation assay was performed with a purified C-terminus fragment of ASPP2 (693-1128) containing both MAPK putative phosphorylation sites. When compared to p38 SAPK, MAPK1 was clearly able to phosphorylate the ASPP2 fragment in vitro (FIG. 26D, left and middle panels). The phosphorylated ASPP2 fragment was digested with trypsin and fractioned on a high performance liquid chromatography (HPLC). Each eluted fraction was measured for its radioactivity content (FIG. 26D, right panel). The fractions representing these radioactive peaks were analyzed by mass spectrometry. Of the two radioactive peaks, one represented the linker region between the GST and our ASPP2 fragment and the other corresponded to a fragment of the same mass as that containing the second putative phosphorylation site, namely serine 827. Hence ASPP2 can be phosphorylated at serine 827 by MAPK in vitro. A synthetic peptide encoding amino acids 824-832, with the serine 827 containing a phosphate group, was used as an antigen to raise antibodies. A polyclonal antibody NGH.S4 was purified by affinity column purification. To test the efficacy of the purified phospho-specific antibody, a non-radioactive in vitro phosphorylation assay was performed on the purified GST-ASPP2 fragment (693-1128) with recombinant MAPK1. FIG. 26E shows that the phospho-specific antibody is specific for the ASPP2 fragment phosphorylated in vitro by MAPK. To test whether endogenous ASPP2 could be phosphorylated in vivo, Saos2 cells were grown in low serum for 50 hours to remove all background stimulation of Ras, after which the cells were stimulated with EGF and 20% FCS. Phosphorylated endogenous ASPP2 was detected by the phospho-specific antibody 30 minutes after Ras stimulation (FIG. 26F). ASPP2 phosphorylation was rapid and transient as 3 hours after EGF stimulation phosphorylated ASPP2 was barely detectable.

### EXAMPLE 16

Phosphorylation of ASPP1 and ASPP2 by MAPK is Required for K-rasV12 and RafCX to Stimulate the Activity of ASP-P1 and ASPP2

Knowing that endogenous Ras is necessary for full ASPP2 activity and that Ras can activate ASPP1 and ASPP2 via the Raf-MAPK pathway, we investigated the requirement of endogenous MAPK activity for ASPP2 to stimulate the transactivation function of p53. A known MEK inhibitor, UO126, was used to test its effect on the transactivation function of p53 via ASPP2. Interestingly, the presence of UO126 had a small effect on the transactivation function of p53 alone but it significantly inhibited the transactivation function of p53 when ASPP2 was present (FIG. 27A). This strongly suggested that endogenous MAPK activity was required for ASPP2 full potential in stimulating p53 activity. To investigate the biological implications of ASPP1 and ASPP2 phosphorylation by MAPK, the putative MAPK sites in both ASPP1 and ASPP2 were mutagenised to generate mutants, each one lacking one of the putative MAPK phosphorylation site by converting the serine to an alanine, namely ASPP1 (S671A), ASPP1 (S746A), ASPP2 (S698A) and ASPP2 (S827A).

In conditions where the cells were starved of serum, these mutants had identical activity to wild-type ASPP1 or ASPP2 in their ability to enhance the transactivation function of p53 (data not shown). However, whereas KrasV12 or activated Raf CX were able to stimulate wild-type ASPP2 and ASPP2 (S698A) by 2.5 fold, they were unable to increase the activity of mutant ASPP2 (S827) (data not shown and FIG. 27B). A similar result was observed with K-rasV12 stimulation of ASPP1: wild-type ASPP1 and ASPP1 (S671A) were stimulated by KrasV12 but ASPP1 (S746A) activity did not increase in the presence of K-rasV12 (FIG. 27C). These results provided evidence that phosphorylation at Ser746 of ASPP1 and Ser827 of ASPP2 by MAPK are necessary for K-rasV12 and RafCX to stimulate the activity of p53 via ASPP1 and ASPP2.

### EXAMPLE 17

Ras-Raf-MAPK Pathway Enhances the Stability of ASPP1 and ASPP2

We have consistently observed that ASPP1 and ASPP2 protein levels were increased when co-transfected with oncogenic Ras. Similarly, we have noticed a decrease in ASPP2 protein levels when cells were treated with the MAPK inhibitor UO126. Thus we hypothesised that Ras, via the Ras-MAPK pathway, could enhance the activity of ASPP1 or ASPP2 by increasing their protein levels.

The ASPP2 protein levels in the presence or absence of transfected oncogenic Ras were measured after cycloheximide addition (FIG. 28A). The total protein level of ASPP2 was clearly higher in the presence of H-RasV12. ASPP2 was also more stable when co-transfected with oncogenic Ras: ASPP2 levels had considerably decreased after 6 hours when co-transfected with the vector alone however in the presence of H-RasV12 the protein levels remained almost identical between 1 and 6 hours after cycloheximide treatment. Interestingly, H-RasV12 failed to increase the expression of the phosphorylation mutant ASPP2 (S827A) suggesting that phosphorylation of ASPP2 by MAPK at serine 827 is required for H-RasV12 to increase the ASPP2 protein level (FIG. 28B). Since oncogenic Ras seemed to affect transfected ASPP2 stability via serine 827 phosphorylation, the effect of endogenous MAPK activity on endogenous ASPP2 stability was investigated. To compare the effect of active and inactive MAPK on ASPP2, MCF7 cells were stimulated with EGF to activate the endogenous Ras-Raf-MAPK pathway and subsequently treated with the MEK inhibitor UO126 to prevent MAPK activation. ASPP2 stability was analyzed in the presence of cycloheximide.

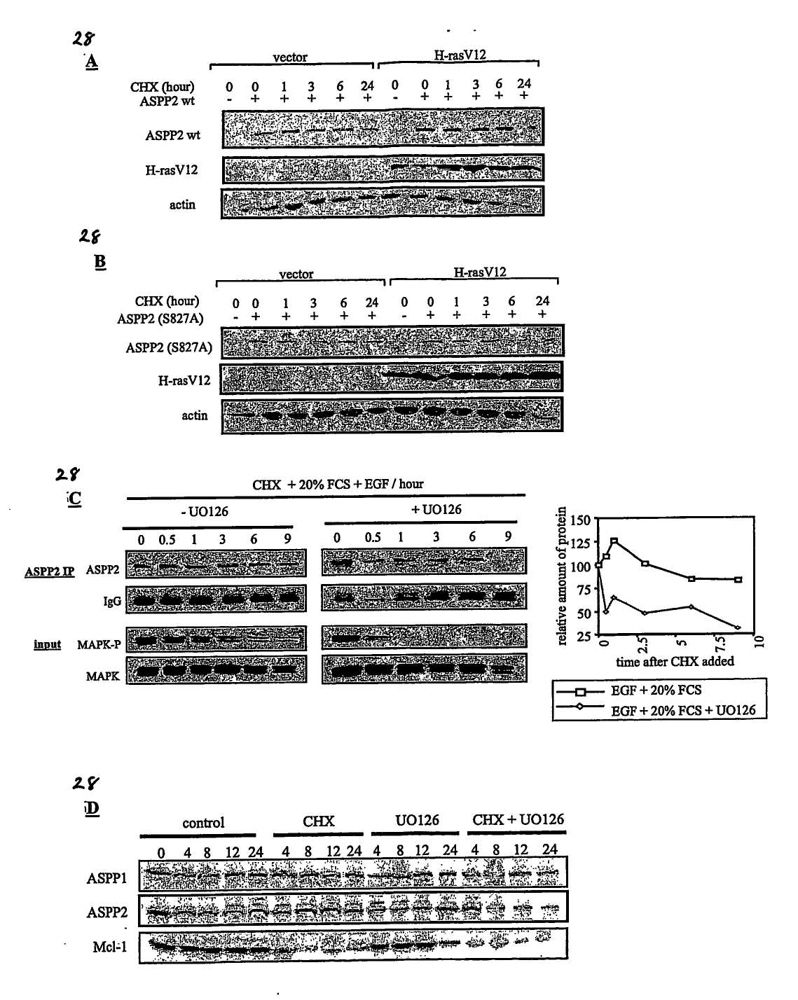

FIG. 28C shows that ASPP2 protein levels were significantly decreased in the presence of UO126. Quantification of the intensity of the bands clearly showed a difference in ASPP2 stability when MAPK was active compared to when MAPK was inactivated by UO126. To confirm the role MAPK plays in increasing ASPP stability, MDA Panc3 cells, containing oncogenic K-rasV1213, were used. ASPP1 and ASPP2 protein levels were monitored in the presence of cycloheximide. Both ASPP1 and ASPP2 expression levels significantly decreased in the presence of cycloheximide and UO126, compared to cycloheximide alone. This confirmed that MAPK activity leads to increased ASPP stability (FIG. 29D). Interestingly, ASPP1 and ASPP2 levels did not seem to decrease much with time in the presence of cycloheximide alone. This might be due to the fact that the oncogenic Ras in these cells results in high MAPK activity, leading to very stable ASPP proteins. Mcl-1 was used as a control for the efficacy of cycloheximide and no difference in its stability was observed following cycloheximide treatment in the presence or absence of UO126

1. A screening method for the identification of agents which modulate, either directly or indirectly, the interaction of a first polypeptide encoded by a nucleic acid molecule selected from the group consisting of:
a) a polypeptide encoded by a nucleic acid molecule comprising a nucleic acid sequence as represented in SEQ ID NO: 1 or 2;
b) a polypeptide encoded by a nucleic acid molecule which hybridises to the nucleic acid molecule in (a) and which enhances the pro-apoptotic activity of p53;
c) a polypeptide encoded by a nucleic acid molecule consisting of a nucleic acid sequence that is degenerate as a result of the genetic code to a nucleic acid molecule as defined in (a) and (b); with a second polypeptide selected from the group consisting of:
d) a polypeptide encoded by a nucleic acid molecule comprising a nucleic acid sequence as represented in SEQ ID NO: 5, 7, 9, or 11;
e) a polypeptide encoded by a nucleic acid molecule which hybridizes to the nucleic acid molecule in (d) and which has the activity associated with Ras or a variant Ras polypeptide;
f) a polypeptide encoded by a nucleic acid molecule consisting of a nucleic acid sequence that is degenerate as a result of the genetic code to a nucleic acid molecule as defined in (d) and (e); the method comprising,
i) forming a preparation comprising said first and second polypeptide;
ii) adding at least one candidate agent to be tested; and
iii) determining the effect, or not, of said agent on the interaction of said first polypeptide with said second polypeptide.

2. The method according to claim 1 wherein said first polypeptide is represented by the amino acid sequence as shown in SEQ ID NO: 3 or 4, or a variant polypeptide wherein said variant polypeptide sequence has been altered by addition, substitution or deletion of at least one amino acid residue.

3. The method according to claim 1 wherein said first polypeptide comprises the amino acid sequence +1 to +120 of the sequence shown in SEQ ID NO: 2 or 3.

4. The method according to claim 3 wherein said polypeptide consists of the amino acid sequence +1 to +120 of the sequence shown in SEQ ID NO: 3 or 4.

5. The method of claim 1 wherein said second polypeptide is represented by the amino acid sequence shown in SEQ ID NO: 6. 8, 10, or 12, or a variant polypeptide wherein said variant polypeptide sequence has been altered by addition, substitution or deletion of at least one amino acid residue.

6. The method according to claim 5 wherein said second polypeptide comprises the amino acid sequence as shown in SEQ ID NO: 8.

7. The method according to claim 5 wherein said second polypeptide comprises the amino acid sequence as shown in SEQ ID NO: 12.

8. The method of claim 1 wherein said second polypeptide is modified at amino acid residue 17.

9. The method according to claim 8 wherein said modification is the substitution of a serine amino acid for an asparagine amino acid.

10. The method of claim 1 wherein said first and second polypeptides are expressed by a cell.

11. The method according to claim 10 wherein said cell is a cell transfected with at least one nucleic acid molecule(s) which encodes said first and second polypeptides.

12. The method according to claim 10 wherein the expression of said nucleic acid molecule(s) is regulatable.

13. The method of claim 10 wherein said cell is a cancer cell.

14. The method of claim 10 wherein said cell is part of a transgenic animal wherein the genome of said animal has been modified to include nucleic acid molecules which encode first and second polypeptides.

15. The method of claim 10 wherein said nucleic acid molecules are expressed in a specific cell/tissue.

16. A screening method for the identification of agents which modulate, either directly or indirectly, the phosphorylation of a first polypeptide encoded by a nucleic acid molecule selected from the group consisting of:
a) a polypeptide encoded by a nucleic acid molecule comprising a nucleic acid sequence as represented in SEQ ID NO: 1 or 2;
b) a polypeptide encoded by a nucleic acid molecule which hybridises to the nucleic acid molecule in (a) and which enhances the pro-apoptotic activity of p53;
c) a polypeptide encoded by a nucleic acid molecule consisting of a nucleic acid sequence that is degenerate as a result of the genetic code to a nucleic acid molecule as defined in (a) and (b); with a second polypeptide selected from the group consisting of:
d) a polypeptide encoded by a nucleic acid molecule comprising a nucleic acid sequence as represented in SEQ ID NO: 13 or 15;
e) a polypeptide encoded by a nucleic acid molecule which hybridizes to the nucleic acid molecule in (d) and which has protein kinase activity;
f) a polypeptide encoded by a nucleic acid molecule consisting of a nucleic acid sequence that is degenerate as a result of the genetic code to a nucleic acid molecule as defined in (d) and (e); comprising,
i) forming a preparation comprising said first and second polypeptide;
ii) adding at least one candidate agent to be tested; and
iii) determining the effect, or not, of said agent on the phosphorylation state of said first polypeptide.

17. A screening method for the identification of agents which modulate, either directly or indirectly, the phosphorylation state of a first polypeptide encoded by a nucleic acid molecule selected from the group consisting of:
a) a polypeptide encoded by a nucleic acid molecule comprising a nucleic acid sequence as represented in SEQ ID NO: 1 or 2;
b) a polypeptide encoded by a nucleic acid molecule which hybridises to the nucleic acid molecule in (a) and which enhances the pro-apoptotic activity of p53;
c) a polypeptide encoded by a nucleic acid molecule consisting of a nucleic acid sequence that is degenerate as a result of the genetic code to a nucleic acid molecule as defined in (a) and (b); with a second polypeptide selected from the group consisting of:
d) a polypeptide encoded by a nucleic acid molecule comprising a nucleic acid sequence as represented in SEQ ID NO: 17;
e) a polypeptide encoded by a nucleic acid molecule which hybridizes to the nucleic acid molecule in (d) and which has protein phosphatase activity;
f) a polypeptide encoded by a nucleic acid molecule consisting of a nucleic acid sequence that is degenerate as a result of the genetic code to a nucleic acid molecule as defined in (d) and (e); the method comprising,
i) forming a preparation comprising said first and second polypeptide;
ii) adding at least one candidate agent to be tested; and
iii) determining the effect, or not, of said agent on the phosphorylation state of said first polypeptide.

18. The method of claim 1 wherein said agent is a polypeptide, peptide, or aptamer.

19. The method according to claim 18 wherein said polypeptide is an antibody, or active binding fragment thereof.

20. The method according to claim 19 wherein said antibody or binding fragment is a monoclonal antibody.

21. The method according to claim 19 wherein said active binding fragment is a single chain antibody variable region fragment or a domain antibody fragment.

22. The method according to claim 19 or wherein said antibody is a humanized or chimeric antibody.

23-24. (canceled)

25. A transfected cell wherein the genome of said cell is modified to consist of include at least one copy of a nucleic acid molecule encoding a polypeptide comprising a nucleic acid sequence as represented in SEQ ID NO: 1 or 2; and at least one copy of a nucleic acid molecule encoding a polypeptide selected from the group consisting of:
(a) a polypeptide encoded by a nucleic acid molecule comprising a nucleic acid sequence as represented in SEQ ID NO: 5, 7, 9, or 11;
(b) a polypeptide encoded by a nucleic acid molecule which hybridizes to the nucleic acid molecule in (a) and which has the activity associated with Ras or a variant Ras polypeptide;
(c) a polypeptide encoded by a nucleic acid molecule consisting of a nucleic acid sequence that is degenerate as a result of the genetic code to a nucleic acid molecule as defined in (a) and
(b) wherein said cell is adapted for the regulated expression of said nucleic acid molecule(s).

26. A cell transfected with at least one nucleic acid molecule wherein the genome of said cell is modified to include at least one copy of a nucleic acid molecule encoding a polypeptide selected from the group consisting of:
a) a polypeptide encoded by a nucleic acid molecule comprising a nucleic acid sequence as represented in SEQ ID NO: 1 or 2;
b) a polypeptide encoded by a nucleic acid molecule which hybridises to the nucleic acid molecule in (a) and which enhances the pro-apoptotic activity of p53;
c) a polypeptide encoded by a nucleic acid molecule consisting of a nucleic acid sequence that is degenerate as a result of the genetic code to a nucleic acid molecule as defined in (a) and (b) and at least one copy of a nucleic acid molecule encoding a polypeptide selected from the group consisting of;
d) a polypeptide encoded by a nucleic acid molecule comprising a nucleic acid sequence as represented in SEQ ID NO: 13 or 15;
e) a polypeptide encoded by a nucleic acid molecule which hybridises hybridizes to the nucleic acid molecule in (d) and which has protein kinase activity;
f) a polypeptide encoded by a nucleic acid molecule consisting of a nucleic acid sequence that is degenerate as a result of the genetic code to a nucleic acid molecule as defined in (d) and (e) wherein said cell is adapted for the regulated expression of said nucleic acid molecule(s).

27. A transfected cell wherein the genome of said cell is modified to consist of at least one copy of a nucleic acid molecule encoding a polypeptide comprising a nucleic acid sequence as represented in SEQ ID NO: 1 or 2;
and at least one copy of a nucleic acid molecule encoding a polypeptide selected from the group consisting of;
(a) a polypeptide encoded by a nucleic acid molecule comprising a nucleic acid sequence as represented in SEQ ID NO: 17;
(b) a polypeptide encoded by a nucleic acid molecule which hybridizes to the nucleic acid molecule in (a) and which has protein phosphatase activity;
(c) a polypeptide encoded by a nucleic acid molecule consisting of a nucleic acid sequence that is degenerate as a result of the genetic code to a nucleic acid molecule as defined in (a) and (b) wherein said cell is adapted for the regulated expression of said nucleic acid molecule(s).

28. The cell of claim 25 wherein said cell further comprises a nucleic acid molecule which includes a reporter gene to monitor the activity of said pro-apoptotic polypeptide(s).

29. The cell according to any of claim 25 wherein said cell is a cancer cell.

30. A non-human transgenic animal comprising at least one cell of claim 25.

31. The animal according to claim 30 wherein said non-human animal is a non-human primate.

32. The animal according to claim 30 wherein said transgenic animal is a rodent or a pig.

33-34. (canceled)

35. A preparation comprising a nucleic acid molecule which encodes a p53 polypeptide, or sequence variant thereof, and at least one nucleic acid molecule which encodes at least one polypeptide, or sequence variant thereof, as represented by the amino acid sequences shown in SEQ ID NO: 3 or 4, wherein said polypeptides are encoded by a single nucleic acid molecule which is part of a vector and said nucleic acid molecules are operably linked to at least one promoter that controls expression of said nucleic acid molecules.

36-38. (canceled)

39. A method to treat a condition which would benefit from an increase in apoptosis comprising administering a preparation comprising a first nucleic acid molecule comprising a nucleic acid sequence which encodes a p53 polypeptide, or sequence variant thereof, and administering a second preparation comprising a second nucleic acid molecule comprising a nucleic acid sequence which encodes a polypeptide, or sequence variant thereof, as represented by the amino acid sequence as shown in SEQ ID NO: 3 or 4 wherein said preparations are administered simultaneously, sequentially or delayed manner.

40. A method to treat a condition which would benefit from a stimulation of apoptosis comprising administering a the preparation of claim 35.

41. The method according to claim 39 wherein said condition is cancer.

42. An antibody, or active binding fragment thereof, wherein said antibody or fragment, specifically binds a polypeptide encoded by a nucleic acid molecule selected from the group consisting of:
a) a polypeptide encoded by a nucleic acid molecule comprising a nucleic acid sequence as represented in SEQ ID NO: 1 or 2;
b) a polypeptide encoded by a nucleic acid molecule which hybridizes to the nucleic acid molecule in (a) and which enhances the pro-apoptotic activity of p53;
c) a polypeptide encoded by a nucleic acid molecule consisting of a nucleic acid sequence that is degenerate as a result of the genetic code to a nucleic acid molecule as defined in (a) and (b), wherein said antibody binds a phosphorylated epitope.

43. The antibody according to claim 42 wherein said antibody is a monoclonal antibody.

44. The antibody according to claim 42 wherein said active binding fragment is a single chain antibody fragment or a domain antibody.

45. The antibody of claim 42 wherein said phosphorylated epitope comprises amino acid residue 671 of the amino acid sequence as shown in SEQ ID NO: 3.

46. The antibody of claim 42 wherein said phosphorylated epitope comprises amino acid residue 698 of the amino acid sequence shown in SEQ ID NO: 4.

47. The antibody of claim 42 wherein said phosphorylated epitope comprises amino acid residue 746 of the amino acid sequence as shown in SEQ ID NO: 3.

48. The antibody of claim 42 wherein said phosphorylated epitope comprises amino acid residue 827 of the amino acid sequence shown in SEQ ID NO: 4.


SEQUENCE LISTING (23 sequences)

SEQ ID NO: 1 - DNA, 4834 bp, Homo sapiens
gagccccgcatcccgccgcagctgccgcctcgccgcggccgggccggagagcacggcggcgggagcgcggccttaggaggcggccggagcggtgggcacagctcggcgcggagcgtcctgtcaggcggcggccgagggcgtcgcggactctccccgcgatgatgccgatgatattaactgttttcttgagcaacaatgaacagattttaacagaagttcctataacaccggaaacaacctgtcgagatgttgtagaattttgcaaggaacctggagaaggcagctgccatttagctgaagtgtggaggggaaatgaacgtcccataccctttgatcatatgatgtacgaacatcttcagatatggggtccacggagggaagaagtgaaatttttccttcgacacgaggactccccaactgagaacagtgaacaaggtggccgtcagacccaagagcaacgaactcagagaaatgtaataaatgtacctggagataaacgtactgaatatggggttgggaatccacgtgttgaacttaccctctcagagctccaagatatggcagctaggcaacagcagcagattgaaaatcagcagcagatgttggttgccaaggaacagcgtttacattttctaaagcaacaggagcgccgtcagcagcagtctatttctgaaaatgaaaagcttcagaaattgaaagaacgagttgaagcccaggagaacaagctgaagaaaattcgtgcaatgagaggacaagtcgactacagcaaaatcatgaacggcaatctgtctgctgaaatagaaaggttcagtgccatgttccaggaaaagaagcaggaagtacagactgcaattttaagggttgatcagcttagtcagcaattggaagatttaaagaaaggaaaactgaatgggttccagtcttacaatggcaaattgacgggaccagcggcggtggagttaaaaagactgtaccaagaactacagattcgtaaccaacttaaccaggaacaaaattcaaaacttcagcagcagaaggaactcttaaataagcgcaacatggaggtggccatgatggacaagcgaatcagtgaactgcgtgaacgtctctatgggaaaaaaattcagctgaaccgtgtgaatggcacgtcatcaccacagtcccctctgagcacatcgggcagggtcgctgctgtggggccttatatccaggttcccagtgccggaagctttcctgtgctgggggaccctataaagccccagtctctcagtattgcctcaaatgctgctcatggaagatccaaatccgctaatgatggaaactggccaacattaaaacagaattctagctcttccgtgaaaccagtgcaggtggccggtgcagactggaaggatccgagcgtggaggggtctgtcaagcagggcactgtctccagccagcctgtgcccttctcagcactgggacccacggagaagccgggcatcgagattggtaaagtgccacctcccatcccgggtgtaggcaagcagctgcctccaagctatgggacatacccaagtcctacacctctgggtcctgggtcgacaagctccctggaaaggaggaaggaaggcagcttgcccaggcccagtgcaggcctgccaagtcgacagaggcccaccctgctgcccgccacaggcagcaccccccagccaggctcctcacaacagattcagcagaggatttccgtaccgccaagtcccacgtacccgccagcgggaccacctgcatttccagctggggacagcaagcctgaactcccactgacagtggccattaggcctttcctggctgataaagggtcaaggccacagtctcccaggaaaggaccccagacagtgaattcaagttccatatactccatgtacctccagcaagccacaccacctaagaattaccagccggcagcacacagcgccttaaataagtcagttaaagcagtgtatggtaagcccgttttaccttcgggttcaacctctccatcgccgctgccgtttcttcacgggtcactgtccacgggcacaccacagcctcagccaccttcagaaagtactgagaaagagcctgagcaggatggccccgccgcccccgcagatggcagcaccgtggagagcctgccacggccactcagccccaccaagctcacgcccatcgtgcattcgccactgcgctaccagagtgatgcagacctggaggccctccgcaggaagctggccaacgcgccccggcccctgaaaaagcgcagctccatcacagagcccgagggccccggcgggcccaacatccagaagctgctgtaccagcgcttcaacaccctggccggtggcatggagggcacccctttctaccagcccagcccctcccaggacttcatgggcaccttggccgatgtggacaatggaaacaccaatgccaatggaaacctggaagagctcccccctgcccagcccacagccccactccccgctgagcctgccccgtcatcagatgccaatgataatgagttaccttcccccgaaccagaggagctcatctgtccccaaaccacccaccaaactgccgagccggcagaggacaataacaacaacgtggccacggtccccaccacggagcagatcccgagtcctgtggctgaggccccatctccaggggaagagcaggtccctccagcacctcttccccctgccagccaccctcctgccacctccacgaacaagcggaccaacttgaagaagcccaactcggagcggacggggcacgggctgagagtccggtttaaccccctggcactgctcctagacgcgtctctggaaggagagttcgatctggtgcagaggatcatctatgaggtggaagatcccagcaagcccaacgatgaagggatcaccccactgcacaacgccgtctgcgccggccaccatcacatcgtgaagttcctgctggattttggtgtcaacgtgaatgctgctgatagtgatggatggacgccgctgcactgcgctgcctcttgtaacagcgttcacctctgcaaacagctggtggagagtggtgccgccatttttgcctcaaccataagcgacattgaaactgctgcagacaagtgtgaggagatggaggaaggctacatccagtgctcccagtttctatatggggtgcaggaaaagctgggtgtgatgaacaaaggtgtggcgtatgctctgtgggactacgaggcccagaacagtgacgagctgtccttccacgaaggggacgccctcaccatcctgaggcgcaaggacgaaagcgagactgagtggtggtgggctcgccttggagaccgggagggctatgtgcccaaaaacctgctggggctgtatccacggatcaaaccccgacagcgaacactcgcctgaacttccttttggagcaccgcatggtcttgccagctaccaggagccacttaagagattattgtgctgttttccaggaaagctgcagctagaaaatggtcttaatggtgctcactttagcagacagcgtccacaatgtgaatcctacagtttccaggtgaggccctttctccagtttgcccattaactgggagaggtactttcgcctccaaggactgaattttgccaattactataaatccaaataaatacccactttcaaaacacccacccctcttgccattaagaagtcccataacccccggttggttgccagtgaagacagaagctcttactgacttggccccgaggccatcaccccctccagcagtgaacactgtccgccgctgtgaggcctgctcccctgcgaccgccctgccccccgtcaccgaatcggacactcatcctttctcacacttcccacacatgatccttcttcccttcatcaccaaaggagcctctgtatggaaacatgtccagtgttgctgcccagtgtgtatgcctcccagtacccactctgctcggccgccttgggggttccgcttcctgttccagttcacctaaaggctgattgtgcaggcccagcactgtggctggactgccgcgccacgggcaccaggacccctaagaccaagtgacaactgggagagcctcagcatatactcttctcctccgatctcacagcctgtcatgctgctcagtgtggttctcacccctgcaagctcaaattcagttccctgaatggagtcaggtgctggaggccgtggcagcggagggtggttggggttggggctgggggtggactggtgtgagggcagaccagggccaggtagacggggctgtttggtgcctgaaggatggcagacgcctggtgtcaggaggggccgccaccaaggagcagcagctggggcagaggagctggggtcaggggccacccctctctgccgatctccctgcctgggctggctgtgaggccacctttgtcccaggcccagcctcaaggcaaggagggcgcttcactgaggtgtgaattgtacgtacaggctttttatataccaaaagtattttttgactagaccattcaaagctacccgaactatgttggaaatttttttttttctcattaaaatacaggcccttaggctctatttttcatgtatgagtcgtgtgtaatttatgtaaaaatgtgtgtacagactcactgatgcagcactgtagcccatcaccttggagcactgactgtacatagtgtggtgaagaaaagtgaacgcccttgtagagcagcccgaccacaggagcatggccgctgccagcccagacgctgctgacgctgtgtaaatgtgcacaataaacccgtctcaccccgg

SEQ ID NO: 2 - DNA, 4402 bp, Homo sapiens
gtcacgagcgtcgaagagacaaagccgcgtcagggggcccggccggggcgggggagcccggggcttgttggtgccccagcccgcgcggagggcccttcggacccgcgcgccgccgctgccgccgccgccgcctcgcaacaggtccgggcggcctcgctctccgctcccctcccccgcatccgcgaccctccggggcacctcagctcggccggggccgcagtctggccacccgcttccatgcggttcgggtccaagatgatgccgatgtttcttaccgtgtatctcagtaacaatgagcagcacttcacagaagttccagttactccagaaacaatatgcagagacgtggtggatctgtgcaaagaacccggcgagagtgattgccatttggctgaagtgtggtgtggctctgaacgtccagttgcggataatgagcgaatgtttgatgttcttcaacgatttggaagtcagaggaacgaagttcgcttcttccttcgtcatgaacgcccccctggcagggacattgtgagtggaccaagatctcaggatccaagtttaaaaagaaatggtgtaaaagttcctggtgaatatcgaagaaaggagaacggtgttaatagtcctaggatggatctgactcttgctgaacttcaggaaatggcatctcgccagcagcaacagattgaagcccagcaacaattgctggcaactaaggaacagcgcttaaagtttttgaaacaacaagatcagcgacaacagcaacaagttgctgagcaggagaaacttaaaaggctaaaagaaatagctgagaatcaggaagctaagctaaaaaaagtgagagcacttaaaggccacgtggaacagaagagactaagcaatgggaaacttgtggaggaaattgaacagatgaataatttgttccagcaaaaacagagggagctcgtcctggctgtgtcaaaagtagaagaactgaccaggcagctagagatgctcaagaacggcaggatcgacagccaccatgacaatcagtctgcagtggctgagcttgatcgcctctataaggagctgcagctaagaaacaaattgaatcaagagcagaatgccaagctacaacaacagagggagtgtttgaataagcgtaattcagaagtggcagtcatggataagcgtgttaatgagctgagggaccggctgtggaagaagaaggcagctctacagcaaaaagaaaatctaccagtttcatctgatggaaatcttccccagcaagccgcgtcagccccaagccgtgtggctgcagtaggtccctatatccagtcgtctactatgcctcggatgccctcaaggcctgaattgctggtgaagccagccctgccggatggttccttggtcattcaggcttcagaggggccgatgaaaatacagacactgcccaacatgagatctggggctgcttcacaaactaaaggctctaaaatccatccagttggccctgattggagtccttcaaatgcagatcttttcccaagccaaggctctgcttctgtacctcaaagcactgggaatgctctggatcaagttgatgatggagaggttccgctgagggagaaagagaagaaagtgcgtccgttctcaatgtttgatgcagtagaccagtccaatgccccaccttcctttggtactctgaggaagaaccagagcagtgaagatatcttgcgggatgctcaggttgcaaataaaaatgtggctaaagtaccacctcctgttcctacaaaaccaaaacagattaatttgccttattttggacaaactaatcagccaccttcagacattaagccagacggaagttctcagcagttgtcaacagttgttccgtccatgggaactaaaccaaaaccagcagggcagcagccgagagtgctgctatctcccagcataccttcggttggccaagaccagaccctttctccaggttctaagcaagaaagtccacctgctgctgccgtccggccctttactccccagccttccaaagacaccttacttccacccttcagaaaaccccagaccgtggcagcaagttcaatatattccatgtatacgcaacagcaggcgccaggaaaaaacttccagcaggctgtgcagagcgcgttgaccaagactcataccagagggccacacttttcaagtgtatatggtaagcctgtaattgctgctgcccagaatcaacagcagcacccagagaacatttattccaatagccagggcaagcctggcagtccagaacctgaaacagagcctgtttcttcagttcaggagaaccatgaaaacgaaagaattcctcggccactcagcccaactaaattactgcctttcttatctaatccttaccgaaaccagagtgatgctgacctagaagccttacgaaagaaactgtctaacgcaccaaggcctctaaagaaacgtagttctattacagagccagagggtcctaatgggccaaatattcagaagcttttatatcagaggaccaccatagcggccatggagaccatctctgtcccatcatacccatccaagtcagcttctgtgactgccagctcagaaagcccagtagaaatccagaatccatatttacatgtggagcccgaaaaggaggtggtctctctggttcctgaatcattgtccccagaggatgtggggaatgccagtacagagaacagtgacatgccagctccttctccaggccttgattatgagcctgagggagtcccagacaacagcccaaatctccagaataacccagaagaaccaaatccagaggctccacatgtgcttgatgtgtacctggaggagtaccctccatacccacccccaccatacccatctggggagcctgaagggcccggagaagactcggtgagcatgcgcccgcctgaaatcaccgggcaggtctctctgcctcctggtaaaaggacaaacttgcgtaaaactggctcagagcgtatcgctcatggaatgagggtgaaattcaacccccttgctttactgctagattcgtctttggagggagaatttgaccttgtacagagaattatttatgaggttgatgacccaagcctccccaatgatgaaggcatcacggctcttcacaatgctgtgtgtgcaggccacacagaaatcgttaagttcctggtacagtttggtgtaaatgtaaatgctgctgatagtgatggatggactccattacattgtgctgcctcatgtaacaacgtccaagtgtgtaagtttttggtggagtcaggagccgctgtgtttgccatgacctacagtgacatgcagactgctgcagataagtgcgaggaaatggaggaaggctacactcagtgctcccaatttctttatggagttcaggagaagatgggcataatgaataaaggagtcatttatgcgctttgggattatgaacctcagaatgatgatgagctgcccatgaaagaaggagactgcatgacaatcatccacagggaagacgaagatgaaatcgaatggtggtgggcgcgccttaatgataaggagggatatgttccacgtaacttgctgggactgtacccaagaattaaaccaagacaaaggagcttggcctgaaacttccacacagaattttagtcaatgaagaattaatctctgttaagaagaagtaatacgattatttttggcaaaaatttcacaagacttattttaatgacaatgtagcttgaaagcgatgaagaatgtctctagaagagaatgaaggattgaagaattcaccattagaggacatttagcgtgatgaaataaagcatctacgtcagcaggccatactgtgttggggcaaaggtgtcccgtgtagcactcagataagtatacagcgacaatcctgttttctacaagaatcctgtctagtaaataggatcatttattgggcagttgggaaatcagctctctgtcctgttgagtgttttcagcagctgctcctaaaccagtcctcctgccagaaaggaccagtgccgtcacatcgctgtctctgattgtccccggcaccagcaggccttggggctcactgaaggctcgaaggcactgcacaccttgtatattgtcagtgaagaacgttagttggttgtcagtgaacaataactttattatatgagtttttgtagcatcttaagaattatacatatgtttgaaatattgaaactaagctacagtaccagtaattagatgtagaatcttgtttgtaggctgaattttaatctgtatttattgtcttttgtatctcagaaattagaaacttgctacagacttacccgtaatatttgtcaagatcatagctgactttaaaaacagttgtaataaactttttgatgct

SEQ ID NO: 3 - protein, 1090 aa, Homo sapiens
MMPMILTVFLSNNEQILTEVPITPETTCRDVVEFCKEPGEGSCHLAEVWRGNERPIPFDHMMYEHLQIWGPRREEVKFFLRHEDSPTENSEQGGRQTQEQRTQRNVINVPGDKRTEYGVGNPRVELTLSELQDMAARQQQQIENQQQMLVAKEQRLHFLKQQERRQQQSISENEKLQKLKERVEAQENKLKKIRAMRGQVDYSKIMNGNLSAEIERFSAMFQEKKQEVQTAILRVDQLSQQLEDLKKGKLNGFQSYNGKLTGPAAVELKRLYQELQIRNQLNQEQNSKLQQQKELLNKRNMEVAMMDKRISELRERLYGKKIQLNRVNGTSSPQSPLSTSGRVAAVGPYIQVPSAGSFPVLGDPIKPQSLSIASNAAHGRSKSANDGNWPTLKQNSSSSVKPVQVAGADWKDPSVEGSVKQGTVSSQPVPFSALGPTEKPGIEIGKVPPPIPGVGKQLPPSYGTYPSPTPLGPGSTSSLERRKEGSLPRPSAGLPSRQRPTLLPATGSTPQPGSSQQIQQRISVPPSPTYPPAGPPAFPAGDSKPELPLTVAIRPFLADKGSRPQSPRKGPQTVNSSSIYSMYLQQATPPKNYQPAAHSALNKSVKAVYGKPVLPSGSTSPSPLPFLHGSLSTGTPQPQPPSESTEKEPEQDGPAAPADGSTVESLPRPLSPTKLTPIVHSPLRYQSDADLEALRRKLANAPRPLKKRSSITEPEGPGGPNIQKLLYQRFNTLAGGMEGTPFYQPSPSQDFMGTLADVDNGNTNANGNLEELPPAQPTAPLPAEPAPSSDANDNELPSPEPEELICPQTTHQTAEPAEDNNNNVATVPTTEQIPSPVAEAPSPGEEQVPPAPLPPASHPPATSTNKRTNLKKPNSERTGHGLRVRFNPLALLLDASLEGEFDLVQRIIYEVEDPSKPNDEGITPLHNAVCAGHHHIVKFLLDFGVNVNAADSDGWTPLHCAASCNSVHLCKQLVESGAAIFASTISDIETAADKCEEMEEGYIQCSQFLYGVQEKLGVMNKGVAYALWDYEAQNSDELSFHEGDALTILRRKDESETEWWWARLGDREGYVPKNLLGLYPRIKPRQRTLA

SEQ ID NO: 4 - protein, 1128 aa, Homo sapiens
MMPMFLTVYLSNNEQHFTEVPVTPETICRDVVDLCKEPGESDCHLAEVWCGSERPVADNERMFDVLQRFGSQRNEVRFFLRHERPPGRDIVSGPRSQDPSLKRNGVKVPGEYRRKENGVNSPRMDLTLAELQEMASRQQQQIEAQQQLLATKEQRLKFLKQQDQRQQQQVAEQEKLKRLKEIAENQEAKLKKVRALKGHVEQKRLSNGKLVEEIEQMNNLFQQKQRELVLAVSKVEELTRQLEMLKNGRIDSHHDNQSAVAELDRLYKELQLRNKLNQEQNAKLQQQRECLNKRNSEVAVMDKRVNELRDRLWKKKAALQQKENLPVSSDGNLPQQAASAPSRVAAVGPYIQSSTMPRMPSRPELLVKPALPDGSLVIQASEGPMKIQTLPNMRSGAASQTKGSKIHPVGPDWSPSNADLFPSQGSASVPQSTGNALDQVDDGEVPLREKEKKVRPFSMFDAVDQSNAPPSFGTLRKNQSSEDILRDAQVANKNVAKVPPPVPTKPKQINLPYFGQTNQPPSDIKPDGSSQQLSTVVPSMGTKPKPAGQQPRVLLSPSIPSVGQDQTLSPGSKQESPPAAAVRPFTPQPSKDTLLPPFRKPQTVAASSIYSMYTQQQAPGKNFQQAVQSALTKTHTRGPHFSSVYGKPVIAAAQNQQQHPENIYSNSQGKPGSPEPETEPVSSVQENHENERIPRPLSPTKLLPFLSNPYRNQSDADLEALRKKLSNAPRPLKKRSSITEPEGPNGPNIQKLLYQRTTIAAMETISVPSYPSKSASVTASSESPVEIQNPYLHVEPEKEVVSLVPESLSPEDVGNASTENSDMPAPSPGLDYEPEGVPDNSPNLQNNPEEPNPEAPHVLDVYLEEYPPYPPPPYPSGEPEGPGEDSVSMRPPEITGQVSLPPGKRTNLRKTGSERIAHGMRVKFNPLALLLDSSLEGEFDLVQRIIYEVDDPSLPNDEGITALHNAVCAGHTEIVKFLVQFGVNVNAADSDGWTPLHCAASCNNVQVCKFLVESGAAVFAMTYSDMQTAADKCEEMEEGYTQCSQFLYGVQEKMGIMNKGVIYALWDYEPQNDDELPMKEGDCMTIIHREDEDEIEWWWARLNDKEGYVPRNLLGLYPRIKPRQRSLA

SEQ ID NO: 5 - DNA, 570 bp, Homo sapiens
atgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctga

SEQ ID NO: 6 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 7 - DNA, 570 bp, Homo sapiens
atgacggaatataagctggtggtggtgggcgccgtcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctga

SEQ ID NO: 8 - protein, 189 aa, Homo sapiens [sequence omitted]

SEQ ID NO: 9 - DNA, 567 bp, Homo sapiens
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 10 - protein, 188 aa, Homo sapiens [sequence omitted]

SEQ ID NO: 11 - DNA, 567 bp, Homo sapiens
atgactgaatataaacttgtggtagttggagcttgcggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 12 - protein, 188 aa, Homo sapiens
MTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 13 - DNA, 1083 bp, Homo sapiens
atggcggcggcggcggcggcgggcgcgggcccggagatggtccgcgggcaggtgttcgacgtggggccgcgctacaccaacctctcgtacatcggcgagggcgcctacggcatggtgtgctctgcttatgataatgtcaacaaagttcgagtagctatcaagaaaatcagcccctttgagcaccagacctactgccagagaaccctgagggagataaaaatcttactgcgcttcagacatgagaacatcattggaatcaatgacattattcgagcaccaaccatcgagcaaatgaaagatgtatatatagtacaggacctcatggaaacagatctttacaagctcttgaagacacaacacctcagcaatgaccatatctgctattttctctaccagatcctcagagggttaaaatatatccattcagctaacgttctgcaccgtgacctcaagccttccaacctgctgctcaacaccacctgtgatctcaagatctgtgactttggcctggcccgtgttgcagatccagaccatgatcacacagggttcctgacagaatatgtggccacacgttggtacagggctccagaaattatgttgaattccaagggctacaccaagtccattgatatttggtctgtaggctgcattctggcagaaatgctttctaacaggcccatctttccagggaagcattatcttgaccagctgaaacacattttgggtattcttggatccccatcacaagaagacctgaattgtataataaatttaaaagctaggaactatttgctttctcttccacacaaaaataaggtgccatggaacaggctgttcccaaatgctgactccaaagctctggacttattggacaaaatgttgacattcaacccacacaagaggattgaagtagaacaggctctggcccacccatatctggagcagtattacgacccgagtgacgagcccatcgccgaagcaccattcaagttcgacatggaattggatgacttgcctaaggaaaagctcaaagaactaatttttgaagagactgctagattccagccaggatacagatcttaa

SEQ ID NO: 14 - protein, 360 aa, Homo sapiens
MAAAAAAGAGPEMVRGQVFDVGPRYTNLSYIGEGAYGMVCSAYDNVNKVRVAIKKISPFEHQTYCQRTLREIKILLRFRHENIIGINDIIRAPTIEQMKDVYIVQDLMETDLYKLLKTQHLSNDHICYFLYQILRGLKYIHSANVLHRDLKPSNLLLNTTCDLKICDFGLARVADPDHDHTGFLTEYVATRWYRAPEIMLNSKGYTKSIDIWSVGCILAEMLSNRPIFPGKHYLDQLKHILGILGSPSQEDLNCIINLKARNYLLSLPHKNKVPWNRLFPNADSKALDLLDKMLTFNPHKRIEVEQALAHPYLEQYYDPSDEPIAEAPFKFDMELDDLPKEKLKELIFEETARFQPGYRS

SEQ ID NO: 15 - DNA, 2685 bp, Homo sapiens
tcgggctgaggttcccgggcgggcgggcgcggagagacgcgggaagcaggggctgggcgggggtcgcggcgccgcagctagcgcagccagcccgagggccgccgccgccgccgcccagcgcgctccggggccgccggccgcagccagcacccgccgcgccgcagctccgggaccggccccggccgccgccgccgcgatgggcaacgccgccgccgccaagaagggcagcgagcaggagagcgtgaaagaattcttagccaaagccaaagaagattttcttaaaaaatgggaaagtcccgctcagaacacagcccacttggatcagtttgaacgaatcaagaccctcggcacgggctccttcgggcgggtgatgctggtgaaacacaaggagaccgggaaccactatgccatgaagatcctcgacaaacagaaggtggtgaaactgaaacagatcgaacacaccctgaatgaaaagcgcatcctgcaagctgtcaactttccgttcctcgtcaaactcgagttctccttcaaggacaactcaaacttatacatggtcatggagtacgtgcccggcggggagatgttctcacacctacggcggatcggaaggttcagtgagccccatgcccgtttctacgcggcccagatcgtcctgacctttgagtatctgcactcgctggatctcatctacagggacctgaagccggagaatctgctcattgaccagcagggctacattcaggtgacagacttcggtttcgccaagcgcgtgaagggccgcacttggaccttgtgcggcacccctgagtacctggcccctgagattatcctgagcaaaggctacaacaaggccgtggactggtgggccctgggggttcttatctatgaaatggccgctggctacccgcccttcttcgcagaccagcccatccagatctatgagaagatcgtctctgggaaggtgcgcttcccttcccacttcagctctgacttgaaggacctgctgcggaacctcctgcaggtagatctcaccaagcgctttgggaacctcaagaatggggtcaacgatatcaagaaccacaagtggtttgccacaactgactggattgccatctaccagaggaaggtggaagctcccttcataccaaagtttaaaggccctggggatacgagtaactttgacgactatgaggaagaagaaatccgggtctccatcaatgagaagtgtggcaaggagttttctgagttttaggggcatgcctgtgcccccatgggttttcttttttcttttttcttttttttggtcgggggggtgggagggttggattgaacagccagagggccccagagttccttgcatctaatttcacccccaccccaccctccagggttagggggagcaggaagcccagataatcagagggacagaaacaccagctgctccccctcatccccttcaccctcctgccccctctcccacttttcccttcctctttccccacagccccccagcccctcagccctcccagcccacttctgcctgttttaaacgagtttctcaactccagtcagaccaggtcttgctggtgtatccagggacagggtatggaaagaggggctcacgcttaactccagcccccacccacacccccatcccacccaaccacaggccccacttgctaagggcaaatgaacgaagcgccaaccttcctttcggagtaatcctgcctgggaaggagagatttttagtgacatgttcagtgggttgcttgctagaatttttttaaaaaaacaacaatttaaaatcttatttaagttccaccagtgcctccctccctccttcctctactcccacccctcccatgtccccccattcctcaaatccattttaaagagaagcagactgactttggaaagggaggcgctggggtttgaacctccccgctgctaatctcccctgggcccctccccggggaatcctctctgccaatcctgcgagggtctaggcccctttaggaagcctccgctctctttttccccaacagacctgtcttcacccttgggctttgaaagccagacaaagcagctgcccctctccctgccaaagaggagtcatcccccaaaaagacagagggggagccccaagcccaagtctttcctcccagcagcgtttccccccaactccttaattttattctccgctagattttaacgtccagccttccctcagctgagtggggagggcatccctgcaaaagggaacagaagaggccaagtccccccaagccacggcccggggttcaaggctagagctgctggggaggggctgcctgttttactcacccaccagcttccgcctcccccatcctgggcgcccctcctccagcttagctgtcagctgtccatcacctctcccccactttctcatttgtgcttttttctctcgtaatagaaaagtggggagccgctggggagccaccccattcatccccgtatttccccctctcataacttctccccatcccaggaggagttctcaggcctggggtggggccccgggtgggtgcgggggcgattcaacctgtgtgctgcgaaggacgagacttcctcttgaacagtgtgctgttgtaaacatatttgaaaactattaccaataaagttttgtttaaaaaaaaaaaaaaaaaa

SEQ ID NO: 16 - protein, 351 aa, Homo sapiens
MGNAAAAKKGSEQESVKEFLAKAKEDFLKKWESPAQNTAHLDQFERIKTLGTGSFGRVMLVKHKETGNHYAMKILDKQKVVKLKQIEHTLNEKRILQAVNFPFLVKLEFSFKDNSNLYMVMEYVPGGEMFSHLRRIGRFSEPHARFYAAQIVLTFEYLHSLDLIYRDLKPENLLIDQQGYIQVTDFGFAKRVKGRTWTLCGTPEYLAPEIILSKGYNKAVDWWALGVLIYEMAAGYPPFFADQPIQIYEKIVSGKVRFPSHFSSDLKDLLRNLLQVDLTKRFGNLKNGVNDIKNHKWFATTDWIAIYQRKVEAPFIPKFKGPGDTSNFDDYEEEEIRVSINEKCGKEFSEF

SEQ ID NO: 17 - DNA, 993 bp, Homo sapiens
atgtccgacagcgagaagctcaacctggactcgatcatcgggcgcctgctggaagtgcagggctcgcggcctggcaagaatgtacagctgacagagaacgagatccgcggtctgtgcctgaaatcccgggagatttttctgagccagcccattcttctggagctggaggcacccctcaagatctgcggtgacatacacggccagtactacgaccttctgcgactatttgagtatggcggtttccctcccgagagcaactacctctttctgggggactatgtggacaggggcaagcagtccttggagaccatctgcctgctgctggcctataagatcaagtaccccgagaacttcttcctgctccgtgggaaccacgagtgtgccagcatcaaccgcatctatggtttctacgatgagtgcaagagacgctacaacatcaaactgtggaaaaccttcactgactgcttcaactgcctgcccatcgcggccatagtggacgaaaagatcttctgctgccacggaggcctgtccccggacctgcagtctatggagcagattcggcggatcatgcggcccacagatgtgcctgaccagggcctgctgtgtgacctgctgtggtctgaccctgacaaggacgtgcagggctggggcgagaacgaccgtggcgtctcttttacctttggagccgaggtggtggccaagttcctccacaagcacgacttggacctcatctgccgagcacaccaggtggtagaagacggctacgagttctttgccaagcggcagctggtgacacttttctcagctcccaactactgtggcgagtttgacaatgctggcgccatgatgagtgtggacgagaccctcatgtgctctttccagatcctcaagcccgccgacaagaacaaggggaagtacgggcagttcagtggcctgaaccctggaggccgacccatcaccccaccccgcaattccgccaaagccaagaaatag

SEQ ID NO: 18 - protein, 330 aa, Homo sapiens
MSDSEKLNLDSIIGRLLEVQGSRPGKNVQLTENEIRGLCLKSREIFLSQPILLELEAPLKICGDIHGQYYDLLRLFEYGGFPPESNYLFLGDYVDRGKQSLETICLLLAYKIKYPENFFLLRGNHECASINRIYGFYDECKRRYNIKLWKTFTDCFNCLPIAAIVDEKIFCCHGGLSPDLQSMEQIRRIMRPTDVPDQGLLCDLLWSDPDKDVQGWGENDRGVSFTFGAEVVAKFLHKHDLDLICRAHQVVEDGYEFFAKRQLVTLFSAPNYCGEFDNAGAMMSVDETLMCSFQILKPADKNKGKYGQFSGLNPGGRPITPPRNSAKAKK

SEQ ID NO: 19 - DNA, 19 bp, Homo sapiens
gttggagctggtggcgtag

SEQ ID NO: 20 - DNA, 9 bp, Homo sapiens
ttcaagaga

SEQ ID NO: 21 - DNA, 19 bp, Homo sapiens
tcaaacgggtgaaggactc

SEQ ID NO: 22 - protein, 7 aa, Homo sapiens
PAPSPGL

SEQ ID NO: 23 - protein, 299 aa, Homo sapiens
QPRVLLSPSIPSVGQDQTSPGSKQESPPAAAVRPFTPQPSKDTLLPPFRKPQTVAASSIYSMYTQQQAPGKNFQQAVQSALTKTHTRGPHFSSVYGKPVIAAAQNQQQHPENIYSNSQGKPGSPEPETEPVSSVQENHENERIPRPLSPTKLLPFLSNPYRNQSDADLEALRKKLSNAPRPLKKRSSITEPEGPNGPNIQKLLYQRTTIAAMETISVPSYPSKSASVTASSESPVEIQNPYLHVEPEKEVVSLVPESLSPEDVGNASTENSDMPAPSPGLDYEPEGVPDNSPNLQNNPE